(19) United States
(12) Patent Application Publication
(10) Pub. No.: US 2010/0286061 A1
(43) Pub. Date: Nov. 11, 2010

(54) PLASMIN-INHIBITORY THERAPIES

(76) Inventors: Laetitia DEVY, Somervile, MA (US); Arthur C. LEY, Newton, MA (US); Robert C. LADNER, Ijamsville, MD (US)

Correspondence Address: LANDO & ANASTASI, LLP, ONE MAIN STREET, SUITE 1100, CAMBRIDGE, MA, 02142, US

(21) Appl. No.: 12/623,954

(22) Filed: Nov. 23, 2009

Related U.S. Application Data
(62) Division of application No. 11/287,121, filed on Nov. 22, 2005, now abandoned.
(60) Provisional application No. 60/630,226, filed on Nov. 22, 2004.

(51) Int. Cl. A61K 38/16; A61P 35/00

(52) U.S. Cl. 514/19.4

(57) ABSTRACT

The disclosure features a method of treating cancers, angiogenesis-related disorders and lymphangiogenesis-related disorders with plasmin inhibitors. An exemplary method includes: administering, to a subject, a plasmin inhibitor, such as a protein that includes a Kunitz domain that inhibits plasmin.

### CROSS-REFERENCE TO RELATED APPLICATIONS

This application claims priority to U.S. Application Ser. No. 60/630,226, filed on Nov. 22, 2004, the content of which are hereby incorporated by reference.

### BACKGROUND

Plasmin is a serine protease predominantly present in the body in its inactive zymogen form (plasminogen). Upon activation, plasmin can process proteins, including zymogens of a matrix metalloproteinase (MMP). The fibrinolytic (plasminogen/plasmin) and matrix metalloproteinase (MMP) proteolytic systems contribute to degradation of ECM and are attractive targets for therapeutic intervention.

### SUMMARY

In one aspect, the disclosure features a method of treating a metastatic or other cancerous disorder. The method includes: administering, to a subject, a plasmin inhibitor, such as a protein that includes a Kunitz domain that inhibits plasmin. In one embodiment, the plasmin inhibitor is one that does not substantially effect hemostasis. In one embodiment, the plasmin inhibitor does not substantially inhibit other proteases. In one embodiment, the Kunitz domain can include at least two polymer moieties (e.g., a polymer moiety attached to each primary amine). In one embodiment, the Kunitz domain can be fused to a carrier protein, e.g., an albumin or a fragment thereof, for example human serum albumin (HSA) or a fragment thereof. The subject can be at risk for, suspected of having, or having the metastatic or other cancerous disorder. For example, the method can include evaluating the subject to determine if a metastatic or potentially metastatic cancer is present. In one embodiment, the cancer cells express high levels of urokinase, which leads to excessive generation of plasmin.

In one embodiment, the Kunitz domain can inhibit plasmin with a Kᵢ of less than 20 nM, 2 nM, or 0.2 nM. The Kunitz domain can have high specificity for plasmin. For example, the Kunitz domain may also inhibit kallikrein with a Kᵢ of between 100 nM to 1 mM, but does not inhibit plasminogen, uPa, or tPa with a Kᵢ of less than 500 nM.

In one embodiment, the Kunitz domain can inhibit LNCAP or HT-1080 cell invasion in vitro and/or inhibit tube formation by endothelial cells in vitro.

In one embodiment, the Kunitz domain includes Xaa1-Xaa2-Xaa3-Xaa4-Cys-Xaa6-Xaa7-Xaa8-Xaa9-Xaa10-Xaa11-Gly-Xaa13-Cys-Xaa15-Xaa16Xaa17-Xaa18-Xaa 19-Arg-Xaa21-Xaa22-Xaa23-Xaa24-Xaa25-Xaa26-Xaa27-Xaa28-Xaa29-Cys-Xaa31-Xaa 32-Phe-Xaa34-Xaa35-Xaa36-Gly-Cys-Xaa39-Xaa40-Xaa41-Xaa42-Xaa43-Xaa44-Xaa45-Xaa46-Xaa47-Xaa48—Xaa49-Xaa50-Cys-Xaa52-Xaa53-Xaa54-Cys-Xaa56-Xaa57-Xaa58 (SEQ ID NO:24). Xaa can be any amino acid (e.g., a non-cysteine amino acid), or at particular positions Xaa can be absent. Useful amino acids at particular positions are described herein. The Kunitz domain can include a human framework region. In one embodiment, the Kunitz domain includes the amino acid sequence of DX-1000. In one embodiment, the Kunitz domain is at least 80% identical to DX-1000. In one embodiment, the Kunitz domain is at least 90% identical to DX-1000. In one embodiment, the Kunitz domain is at least 95% identical to DX-1000. In one embodiment, the Kunitz domain is identical to DX-1000. In one embodiment, the Kunitz domain differs from DX-1000 by fewer than 3 amino acid differences.

In one embodiment, the plasmin inhibitor does not impair coagulation or platelet function, or is administered at a concentration that does not impair coagulation or platelet function. For example, the plasmin inhibitor is at a concentration of less than 700, 500, or 200 nM.

The method can include other features described herein.

In another aspect, the disclosure features a method of treating a cancer, e.g., a fibrosarcoma, a fibrosarcoma-derived metastasis, a prostate cancer, a prostate cancer-derived metastasis, a breast cancer, a breast cancer-derived metastatis, an angiogenesis-dependent cancer, an angiogenesis-dependent cancer derived metastasis, a lymphangiogenesis-related cancer or other cancer described herein. The method includes: administering, to a subject, an effective amount of a protein that inhibits plasmin. For example, the protein includes a Kunitz domain that inhibits plasmin.

The method can further include administering, to the subject, a second anti-cancer agent. For example, the second anti-cancer agent is leuprolide, goserelin, flutamide, bicalutamide, nilutamide, ketoconazole or aminoglutethimide. The method can include other features described herein.

The method can further include administering, to the subject, plasma kallikrein inhibitor, for example DX-88. The method can include other features described herein.

In another aspect, the disclosure features a method of administering a plasmin inhibitor described herein as an adjuvant therapy, e.g., to a subject. The adjuvant therapy can be a post-operative therapy that is administered to the subject after the subject has undergone surgery to remove all or part of a tumor (e.g., after surgery to treat prostate or breast or angiogenesis-dependent cancer). For example, the plasmin inhibitor is a protein that inhibits plasmin, e.g., a protein that includes a Kunitz domain. In one embodiment, the plasmin inhibitor is administered within 6, 12, 24, 48, or 100 hours of surgery. The plasmin inhibitor can be administered before, during, as well as after surgery. The method can include other features described herein.

In another aspect, the disclosure features a method of treating a disorder attributable to excessive plasmin activity. The method includes administering, to a human or animal subject, a plasmin-inhibitory amount of a protein including a Kunitz domain that inhibits plasmin. For example, the protein includes at least two polymer moieties. The protein can include DX-1000 and three or four PEG moieties. In one embodiment, the protein is one that does not substantially effect hemostasis. In one embodiment, the protein does not substantially inhibit other proteases. The method can include other features described herein.

In another aspect, the disclosure features a method of treating a disorder attributable to excessive plasmin activity. The method includes administering, to a human or animal subject, a plasmin-inhibitory amount of a protein including a Kunitz domain that inhibits plasmin. For example, the protein includes DX-1000 fused to albumin, or a fragment thereof. In one embodiment, the protein is one that does not substantially effect hemostasis. In one embodiment, the protein does not substantially inhibit other proteases. The method can include other features described herein.

In another aspect, the disclosure features a method that includes: evaluating a subject for risk or presence of a cancer (e.g., a metastatic cancer); and if an indication of cancer (particularly metastatic cancer) is detected, administering to the subject, an effective amount of a protein including a Kunitz domain that inhibits plasmin. In one embodiment, the cancer is prostate cancer or another cancer disclosed herein. For example, the step of evaluating can include detecting a prostate-specific antigen in a sample from the subject. The step of evaluating can include administering to the subject a reagent that binds to a prostate-specific antigen, and imaging the subject. The method can include other features described herein.

In another aspect, the disclosure features a method of inhibiting angiogenesis in a subject. In one embodiment, the method includes: administering, to a subject, a plasmin inhibitor, such as a protein that includes a Kunitz domain that inhibits plasmin, wherein the Kunitz domain includes at least two polymer moieties. For example, the protein includes DX-1000 and three or four PEG moieties. For example, the protein includes DX-1000 fused to human serum albumin (HSA) or a fragment thereof. In one embodiment, the plasmin inhibitor is one that does not substantially effect hemostasis. In one embodiment, the plasmin inhibitor does not substantially inhibit other proteases. The method can include other features described herein.

In another aspect, the disclosure features a method of treating an angiogenesis-related disorder, e.g., an ocular angiogenic disease, inflammation, or an angiogenesis-dependent cancer or tumor. The method includes: administering, to a subject, an effective amount of a plasmin inhibitor, such as a protein that inhibits plasmin. For example, the protein includes a Kunitz domain that inhibits plasmin. For example, the protein includes at least two polymer moieties. The protein can include DX-1000 and three or four PEG moieties. In one embodiment, the protein is one that does not substantially effect hemostasis. In one embodiment, the protein does not substantially inhibit other proteases. The method can include other features described herein.

In another aspect, the disclosure features a method of treating lymphangiogenesis-related disorder, e.g., cancer, e.g. metastatic cancer, e.g., metastatic breast, ovarian or colorectal cancer. In one embodiment, the method includes administering, to a human or animal subject, a plasmin-inhibitory amount of a protein including a Kunitz domain that inhibits plasmin. For example, the protein includes at least two polymer moieties. The protein can include DX-1000 and three or four PEG moieties. In one embodiment, the protein is one that does not substantially effect hemostasis. In one embodiment, the protein does not substantially inhibit other proteases. The method can include other features described herein.

In another aspect, the disclosure features a method of reducing VEGF-C and/or VEGF-D activity in a subject. In one embodiment, the method includes administering, to a human or animal subject, a plasmin-inhibitory amount of a protein including a Kunitz domain that inhibits plasmin, e.g., DX-1000. The method can include other features described herein.

It is understood that a protein described herein (e.g., a protein that includes a Kunitz domain) may have mutations relative to a particular protein described herein (e.g., a conservative or non-essential amino acid substitutions), which do not have a substantial effect on the protein function (e.g., ability to inhibit plasmin). Whether or not a particular substitution will be tolerated, i.e., will not adversely affect desired biological properties, such as binding activity, can be determined as described in Bowie, et al. (1990) Science 247:1306-1310. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). It is possible for many framework and CDR amino acid residues to include one or more conservative substitutions.

The comparison of sequences and determination of percent identity between two sequences can be accomplished using a mathematical algorithm. Typically, the percent identity between two nucleotide sequences is determined using the GAP program in the GCG software package, using a Blossum 62 scoring matrix with a gap penalty of 12, a gap extend penalty of 4, and a frameshift gap penalty of 5.

A “non-essential” amino acid residue is a residue that can be altered from the wild-type sequence of the binding agent, e.g., the antibody, without abolishing or more preferably, without substantially altering a biological activity, whereas an “essential” amino acid residue results in such a change.

The term “alkyl” refers to a hydrocarbon chain that may be a straight chain or a branched chain, containing the indicated number of carbon atoms. For example, C₁-C₁₂ alkyl indicates that the group may have from 1 to 12 (inclusive) carbon atoms in it.

The term “aryl” refers to an aromatic monocyclic, bicyclic, or tricyclic hydrocarbon ring system, wherein any ring atom capable of substitution can be substituted by a substituent. Examples of aryl moieties include, but are not limited to, phenyl, naphthyl, and anthracenyl.

Binding affinities can be determined using BIA-CORE analysis, or comparable assay, in phosphate buffered saline at pH 7.2.

The details of one or more embodiments of the invention are set forth in the accompanying drawings and the description below. Other features, objects, and advantages of the invention will be apparent from the description and drawings, and from the claims. All published patent applications, patents, and references cited herein are incorporated by reference in their entirety. In particular, U.S. Pat. Nos. 5,663,143; 5,223,409, 6,010,080, 6,103,499 and 6,333,402 and U.S. Ser. No. 10/931,153 are incorporated by reference in their entireties.

### DESCRIPTION OF THE DRAWINGS

FIG. 1 is a schematic illustrating the function of plasmin in relation to other proteins.

FIG. 2 is a graph showing change in cell migration in the presence and absence of DX-1000.

FIG. 3 is a graph showing the inhibitory effect of DX-1000 on LNCaP cell invasion.

FIGS. 4 & 5 are graphs of the inhibitory effect of DX-1000 on HT-1080 cell invasion.

FIGS. 6 & 7 are graphs comparing the inhibitory effect of DX-1000 and 4-PEG DX-1000 on LNCaP cells (FIG. 6) and HT-1080 cells (FIG. 7).

FIG. 8 is a graph showing biodistribution of DX-1000 and 4-PEGS-DX-1000 in normal mice.

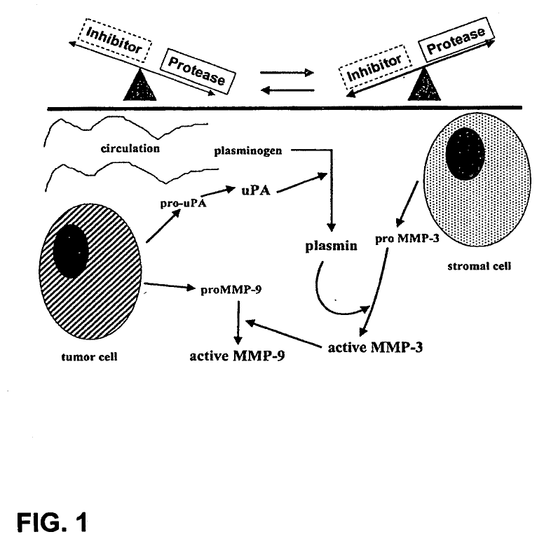

### DETAILED DESCRIPTION

Plasmin (an activated form of plasminogen) is a serine protease important in fibrinolysis. Plasmin is also the key enzyme in angiogenesis, or vascular remodeling. Inhibitors of plasmin can be used to prevent or reduce metastasis of neoplastic cells, e.g., by inhibiting vascular remodeling, alterations to the extracellular matrix and other mechanisms. Vascular remodeling produces lasting structural changes in the vessel wall in response to hemodynamic stimuli. Vascular remodeling is a component of many pathophysiological processes requiring degradation of extracellular matrix (ECM), cell proliferation and migration. Methods of inhibiting this remodeling process can be used to treat neoplastic disorders, particularly disorders related to metastatic cancer. Exemplary cancers include prostate cancer, breast cancer, ovarian cancer, colorectal cancer and fibroscarcomas. Other relevant cancers can include cancers derived from lung, lymphoid, gastrointestinal (e.g., colon), and genitourinary tract, ovary, and pharynx.

Exemplary plasmin inhibitors include DX-1000 and other proteins that include a Kunitz domain.

### DX-1000

DX-1000 is a Kunitz domain that inhibits a human plasmin inhibitor (Kᵢ=88 μM). Further description of Kunitz domains is provided below. The DX-1000 protein includes the framework region of human LACI, but other frameworks can also be used. The sequence of DX-1000 can include the following amino acid sequence (SEQ ID NO:22):

```
        5 10 15 20 25 30 35 40
        . . . . . . . .
MHSFCAFKAETGPCRARFDRWFFNIFTRQCEEFIYGGCEGNQNR
45 50 55
. . .
FESLEECKKMCTRD
```

The sequence can also be preceded by two N-terminal amino acids (“EA”) to include the following sequence (SEQ ID NO:23):

EAMHSFCAFKAETGPCRARFDRWFFNIFTRQCEEFIYGGCEGNQNRFE
SLEECKKMCTRD

DX-1000 was tested in several functional cell-based activity assays and demonstrated potent inhibitory activity. Firstly, DX-1000 (1 nM) inhibited both DHT-stimulated invasion of LNCaPs (prostate cancer) and HT-1080 (fibrosarcoma) through Matrigel, processes known to be dependent on the plasminogen/plasmin system. Interestingly, DX-1000 down-regulated efficiently the expression and activation of gelatinases, directly involved in cancer cell invasion and ECM proteolysis. In addition, DX-1000 (1-10 nM) efficiently blocked tube formation of human and mouse endothelial cells whether plated on Matrigel or collagen type I. Concerning the haemostatic aspect, DX-1000 showed no clinically significant effects on global coagulation screening tests or a platelet function screening test.

DX-1000 can be modified, e.g., by pegylation. It has a three available lysines and an N-terminus for modification with mPEG, one, two, or more of these positions can be modified (e.g., all four of these positions can be modified). The compound 4×PEG DX-1000 is an exemplary modified DX-1000 molecule that includes four PEG moieties. DX-1000 can be combined with an mPEG succinimidyl propionic acid reagent having an average molecular weight of about 5 kDa or 7 kDa.

DX-1000 can also be fused to albumin, or a fragment thereof, to extend its in vivo half-life, therapeutic activity, or shelf-life. The albumin fusion protein can comprise albumin (for example, human serum albumin), or a fragment thereof, as the N-terminal portion, and DX-1000 as the C-terminal portion. Alternatively, an albumin fusion protein may comprise albumin (for example, human serum albumin), or a fragment thereof, as the C-terminal portion, and DX-1000 as the N-terminal portion. In addition, DX-1000 may also be inserted into an internal region of albumin (for example, human serum albumin), or a fragment thereof.

### DX-1000 Variants and Other Inhibitory Kunitz Domains

U.S. Pat. No. 6,103,499 describes additional plasmin inhibitors, including a variety of Kunitz domains, including QS4, NS4, SPI11, SPI15, SPI 08, SPI23, SPI22, SPI60, SPI43, SPI51, SPI54, SPI47, SPI41, DPI-1.1.1, DPI-1.1.2, DPI-1.1.6 and others. The patent also shows examples of variations at particular positions in the binding loop and describes DX-1000 variants that inhibit plasmin. The Kunitz domains described in the patent can also be used in the therapeutic methods described herein.

Exemplary plasmin-inhibitory amount of a protein comprising a Kunitz domain having the formula:

(SEQ ID NO: 24)
Xaa1-Xaa2-Xaa3-Xaa4-Cys-Xaa6-Xaa7-Xaa8-Xaa9-Xaa10-
Xaa11-Gly-Xaa13-Cys-Xaa15-Xaa16Xaa17-Xaa18-Xaa19-
Arg-Xaa21-Xaa22-Xaa23-Xaa24-Xaa25-Xaa26-Xaa27-
Xaa28-Xaa29-Cys-Xaa31-Xaa 32-Phe-Xaa34-Xaa35-
Xaa36-Gly-Cys-Xaa39-Xaa40-Xaa41-Xaa42-Xaa43-Xaa44-
Xaa45-Xaa46-Xaa47-Xaa48-Xaa49-Xaa50-Cys-Xaa52-
Xaa53-Xaa54-Cys-Xaa56-Xaa57-Xaa58.

Xaa1, Xaa2, Xaa3, Xaa4, Xaa56, Xaa57 and/or Xaa58 may be absent. Xaa10 can be Asp, Glu, Tyr, or Gln. Xaa11 can be Thr, Ala, Ser, Val or Asp. Xaa13 can be Pro, Leu or Ala. Xaa15 can be Lys or Arg. Xaa16 can be Ala or Gly. Xaa17 can be Arg, Lys or Ser. Xaa18 can be Phe or Ile. Xaa19 can be Glu, Gln, Asp, Pro, Gly, Ser or Ile. Xaa21 can be Phe, Tyr or Trp. Xaa22 can be Tyr or Phe. Xaa23 can be Tyr or Phe. Xaa31 can be Asp, Glu, Thr, Val, Gln or Ala. Xaa32 can be Thr, Ala, Glu, Pro, or Gln. Xaa34 can be Val, Ile, Thr, Leu, Phe, Tyr, H is, Asp, Ala, or Ser. Xaa35 can be Tyr or Trp. Xaa36 can be Gly or Ser. Xaa39 can be Glu, Gly, Asp, Arg, Ala, Gln, Leu, Lys, or Met. Xaa40 can be Gly or Ala. Xaa43 can be Asn or Gly; or Xaa45 can be Phe or Tyr. Where not specified, Xaa can be any amino acid, particularly any non-cysteine amino acid.

Further, Xaa10 can be Asp or Glu. Xaa11 can be Thr, Ala, or Ser. Xaa13 is Pro. Xaa15 is Arg. Xaa16 is Ala. Xaa17 is Arg. Xaa18 is Phe. Xaa19 can be Glu or Asp. Xaa21 can be Phe or Trp. Xaa22 can be Tyr or Phe. Xaa23 can be Tyr or Phe. Xaa31 can be Asp or Glu. Xaa32 can be Thr, Ala, or Glu. Xaa34 can be Val, Ile or Thr. Xaa35 is Tyr. Xaa36 is Gly. Xaa39 can be Glu, Gly, or Asp. Xaa40 can be Gly or Ala. Xaa43 can be Asn or Gly; or Xaa45 can be Phe or Tyr.

In one embodiment, the protein includes at least 80, 85, 90, 95%, or 100% of the amino acid sequence of the first and/or second binding loops of DX-1000. In one embodiment, the protein includes a framework region from a human Kunitz domain (e.g., a human Kunitz domain described herein).

Exemplary DX-1000 variants include proteins that have an amino acid sequence that differs by at least one, but fewer than eight, six, five, four, three, or two amino acid differences (e.g., substitutions, insertions, or deletions) from the amino acid sequence of DX-1000 (e.g., SEQ ID NO:23) or the amino acid sequence of SEQ ID NO:22. The differences may be in regions other than the first binding loop, or in regions other than the first and second binding loops, e.g., in the framework region. Typically, the Kunitz domain does not naturally occur in humans, but may include an amino acid sequence that differs by fewer than ten, seven, or four amino acids from a human Kunitz domain (e.g., a human Kunitz domain described herein).

In one embodiment, the Kᵢ of the compound for plasmin is within a factor of 0.5 to 1.5, 0.8 to 1.2, 0.3 to 3.0, 0.1 to 10.0, or 0.02 to 50.0 of the Kᵢ of DX-1000 for plasmin.

### Kunitz Domains

DX-1000 includes a Kunitz domain that inhibits plasmin. DX-1000 and related Kunitz domains are described herein.

A Kunitz domain is a polypeptide domain having at least 51 amino acids and containing at least two, and preferably three, disulfides. The domain is folded such that the first and sixth cysteines, the second and fourth, and the third and fifth cysteines form disulfide bonds (e.g., in a Kunitz domain having 58 amino acids, cysteines can be present at positions corresponding to amino acids 5, 14, 30, 38, 51, and 55, according to the number of the BPTI sequence provided below, and disulfides can form between the cysteines at position 5 and 55, 14 and 38, and 30 and 51), or, if two disulfides are present, they can form between a corresponding subset of cysteines thereof. The spacing between respective cysteines can be within 7, 5, 4, 3 or 2 amino acids of the following spacing between positions corresponding to: 5 to 55, 14 to 38, and 30 to 51, according to the numbering of the BPTI sequence provided below. The BPTI sequence can be used as a reference to refer to specific positions in any generic Kunitz domain. Comparison of a Kunitz domain of interest to BPTI can be performed by identifying the best-fit alignment in which the number of aligned cysteines is maximized.

The 3D structure (at high resolution) of the Kunitz domain of BPTI is known. One of the X-ray structures is deposited in the Brookhaven Protein Data Bank as “6PTI”. The 3D structure of some BPTI homologues (Eigenbrot et al., (1990) Protein Engineering, 3(7):591-598; Hynes et al., (1990) Biochemistry, 29:10018-10022) are known. At least seventy Kunitz domain sequences are known. Known human homologues include three Kunitz domains of LACI (Wun et al., (1988) J. Biol. Chem. 263(13):6001-6004; Girard et al., (1989) Nature, 338:518-20; Novotny et al., (1989) J. Biol. Chem., 264(31):18832-18837), two Kunitz domains of Inter-α-Trypsin Inhibitor, APP-I (Kido et al., (1988) J. Biol. Chem., 263(34):18104-18107), a Kunitz domain from collagen, and three Kunitz domains of TFPI-2 (Sprecher et al., (1994) PNAS USA, 91:3353-3357). LACI is a human serum phosphoglycoprotein with a molecular weight of 39 kDa (amino acid sequence in Table 1) containing three Kunitz domains.

TABLE 1
Exemplary Natural Kunitz Domains

| | |
|---|---|
| LACI: | 1 MIYTMKKVHA LWASVCLLLN LAPAPLNAds eedeehtiit dtelpplklM |
| (SEQ ID NO. 1) | 51 HSFCAFKADD GPCKAIMKRF FFNIFTRQCE EFIYGGCEGN QNRFESLEEC |
| | 101 KKMCTRDnan riikttlqqe kpdfCfleed pgiCrgyitr yfynnqtkqC |
| | 151 erfkyggClg nmnnfetlee CkniCedgpn gfqvdnygtq lnavnnsltp |
| | 201 qstkvpslfe fhgpswCltp adrglCrane nrfyynsvig kCrpfkysgC |
| | 251 ggnennftsk geClraCkkg fiqriskggl iktkrkrkkq rvkiayeeif |
| | 301 vknm |
| | The signal sequence (1-28) is uppercase and underscored |
| | LACI-K1 is uppercase |
| | LACI-K2 is underscored |
| | LACI-K3 is bold |
| BPTI | 1 2 3 4 5 |
| (SEQ ID NO: 2) | 1234567890123456789012345678901234567890123456789012345678 |
| | RPDFCLEPPYTGPCKARIIRYFYNAKAGLCQTFVYGGCRAKRNNFKSAEDCMRTCGGA |

The Kunitz domains above are referred to as LACI-K1 (residues 50 to 107), LACI-K2 (residues 121 to 178), and LACI-K₃ (213 to 270). The cDNA sequence of LACI is reported in Wun et al. (J. Biol. Chem., 1988, 263(13):6001-6004). Girard et al. (Nature, 1989, 338:518-20) reports mutational studies in which the P1 residues of each of the three Kunitz domains were altered. LACI-K1 inhibits Factor VIIa (F.VIIa) when F.VIIa is complexed to tissue factor and LACI-K2 inhibits Factor Xa.

Proteins containing exemplary Kunitz domains include the following, with SWISS-PROT Accession Numbers in parentheses:

| A4_HUMAN | (P05067), | A4_MACFA | (P53601), |
|---|---|---|---|
| A4_MACMU | (P29216), | A4_MOUSE | (P12023), |
| A4_RAT | (P08592), | A4_SAISC | (Q95241), |
| AMBP_PLEPL | (P36992), | APP2_HUMAN | (Q06481), |
| APP2_RAT | (P15943), | AXP1_ANTAF | (P81547), |
| AXP2_ANTAF | (P81548), | BPT1_BOVIN | (P00974), |
| BPT2_BOVIN | (P04815), | CA17_HUMAN | (Q02388), |
| CA36_CHICK | (P15989), | CA36_HUMAN | (P12111), |
| CRPT_BOOMI | (P81162), | ELAC_MACEU | (062845), |
| ELAC_TRIVU | (Q29143), | EPPI_HUMAN | (095925), |
| EPPI_MOUSE | (Q9DA01), | HTIB_MANSE | (P26227), |
| IBP_CARCR | (P00993), | IBPC_BOVIN | (P00976), |
| IBPI_TACTR | (P16044), | IBPS_BOVIN | (P00975), |
| ICS3_BOMMO | (P07481), | IMAP_DROFU | (P11424), |
| IP52_ANESU | (P10280), | ISC1_BOMMO | (P10831), |
| ISC2_BOMMO | (P10832), | ISH1_STOHE | (P31713), |
| ISH2_STOHE | (P81129), | ISIK_HELPO | (P00994), |
| ISP2_GALME | (P81906), | IVB1_BUNFA | (P25660), |
| IVB1_BUNMU | (P00987), | IVB1_VIPAA | (P00991), |
| IVB2_BUNMU | (P00989), | IVB2_DABRU | (P00990), |
| IVB2_HEMHA | (P00985), | IVB2_NAJNI | (P00986), |
| IVB3_VIPAA | (P00992), | IVBB_DENPO | (P00983), |
| IVBC_NAJNA | (P19859), | IVBC_OPHHA | (P82966), |
| IVBE_DENPO | (P00984), | IVBI_DENAN | (P00980), |
| IVBI_DENPO | (P00979), | IVBK_DENAN | (P00982), |
| IVBK_DENPO | (P00981), | IVBT_ERIMA | (P24541), |
| IVBT_NAJNA | (P20229), | MCPI_MELCP | (P82968), |
| SBPI_SARBU | (P26228), | SPT3_HUMAN | (P49223), |
| TKD1_BOVIN | (Q28201), | TKD1_SHEEP | (Q29428), |
| TXCA_DENAN | (P81658), | UPTI_PIG | (Q29100), |
| AMBP_BOVIN | (P00978), | AMBP_HUMAN | (P02760), |
| AMBP_MERUN | (Q62577), | AMBP_MESAU | (Q60559), |
| AMBP_MOUSE | (Q07456), | AMBP_PIG | (P04366), |
| AMBP_RAT | (Q64240), | IATR_HORSE | (P04365), |
| IATR_SHEEP | (P13371), | SPT1_HUMAN | (043278), |
| SPT1_MOUSE | (Q9R097), | SPT2_HUMAN | (043291), |
| SPT2_MOUSE | (Q9WU03), | TFP2_HUMAN | (P48307), |
| TFP2_MOUSE | (035536), | TFPI_HUMAN | (P10646), |
| TFPI_MACMU | (Q28864), | TFPI_MOUSE | (054819), |
| TFPI_RABIT | (P19761), | TFPI_RAT | (Q02445), |
| YN81_CAEEL | (Q03610) | | |

TABLE 2
The amino-acid sequences of 19 human Kunitz domains.
Amino-acid sequences of 19 Human Kunitz Domains Binding loops are underscored.

Collagen A1 VII (SEQ ID NO:3)
SDDPCSLPLDEGSCTAYTLRWYHRAVTEACHPFVYGGCGGNANRFGTREACERRCPPR
TFPI2-K1(SEQ ID NO: 4)
NAEICLLPLDYGPCRALLLRYYYDRYTQSCRQFLYGGCEGNANNFYTWEACDDACWRI
AppI (SEQ ID NO: 5)
VREVCSEQAETGPCRAMISRWYFDVTEGKCAPFFYGGCGGNRNNFDTEEYCMAVCGSA
Hep GF AI T2, K2 (SEQ ID NO: 6)
YEEYCTANAVTGPCRASFPRWYFDVERNSCNNFIYGGCRGNKNSYRSEEACMLRCFRQ
ITI, Kl (SEQ ID NO: 7)
KEDSCQLGYSAGPCMGMTSRYFYNGTSMACETFQYGGCMGNGNNFVTEKECLQTCRTV
Chrome20 (SEQ ID NO: 8)
FQEPCMLPVRHGNCNHEAQRWHFDFKNYRCTPFKYRGCEGNANNFLNEDACRTACMLIR
Hep GF AI T1, K1 (SEQ ID NO: 9)
TEDYCLASNKVGRCRGSFPRWYYDPTEQICKSFVYGGCLGNKNNYLREEECILACRGV
Hep GF AI T1, K2 (SEQ ID NO: 10)
DKGHCVDLPDTGLCKESIPRWYYNPFSEHCARFTYGGCYGNKNNFEEEQQCLESCRGI
TFPI2-K3 (SEQ ID NO: 11)
IPSFCYSPKDEGLCSANVTRYYFNPRYRTCDAFTYTGCGGNDNNFVSREDCKRACAKA
ITI, K2 (SEQ ID NO: 12)
AACNLPIVRGPCRAFIQLWAFDAVKGKCVLFPYGGCQGNGNKFYSEKECREYCGVP
Hep GF AI T2, K1 (SEQ ID NO: 13)
IHDFCLVSKVVGRCRASMPRWWYNVTDGSCQLFVYGGCDGNSNNYLTKEECLKKCATV
App2 (SEQ ID NO: 14)
VKAVCSQEAMTGPCRAVMPRWYFDLSKGKCVRFIYGGCGGNRNNFESEDYCMAVCKAM
TFPI1 K2 = LACI-D2 (SEQ ID NO: 15)
KPDFCFLEEDPGICRGYITRYFYNNQTKQCERFKYGGCLGNMNNFETLEECKNICEDG
TFPI2-K2 (SEQ ID NO: 16)
VPKVCRLQVSVDDQCEGSTEKYFFNLSSMTCEKFFSGGCHRNRIENRFPDEATCMGFCAPK
HKI B9 (SEQ ID NO: 17)
LPNVCAFPMEKGPCQTYMTRWFFNFETGECELFAYGGCGGNSNNFLRKEKCEKFCKFT
TFPI1 K1 = LACI-D1 (SEQ ID NO: 18)
MHSFCAFKADDGPCKAIMKRFFFNIFTRQCEEFIYGGCEGNQNRFESLEECKKMCTRD
TFPI1 K3 = LACI-D3 (SEQ ID NO: 19)
GPSWCLTPADRGLCRANENRFYYNSVIGKCRPFKYSGCGGNENNFTSKQECLRACKKG
Collagen A3 (SEQ ID NO: 20)
ETDICKLPKDEGTCRDFILKWYYDPNTKSCARFWYGGCGGNENKFGSQKECEKVCAPV
CAB37635 (SEQ ID NO: 21)
KQDVCEMPKETGPCLAYFLHWWYDKKDNTCSMFVYGGCQGNNNNFQSKANCLNTCKNK
End Table 2.

A variety of methods can be used to identify a Kunitz domain from a sequence database. For example, a known amino acid sequence of a Kunitz domain, a consensus sequence, or a motif (e.g., the ProSite Motif) can be searched against the GenBank sequence databases (National Center for Biotechnology Information, National Institutes of Health, Bethesda Md.), e.g., using BLAST; against Pfam database of HMMs (Hidden Markov Models) e.g., using default parameters for Pfam searching; against the SMART database; or against the Propom database. For example, the Pfam Accession Number PF00014 of Pfam Release 9 provides numerous Kunitz domains and an HMM for identify Kunitz domains. A description of the Pfam database can be found in Sonhammer et al. (1997) Proteins 28(3):405-420 and a detailed description of HMMs can be found, for example, in Gribskov et al. (1990) Meth. Enzymol. 183:146-159; Gribskov et al. (1987) Proc. Natl. Acad. Sci. USA 84:4355-4358; Krogh et al. (1994) J. Mol. Biol. 235:1501-1531; and Stultz et al. (1993) Protein Sci. 2:305-314. The SMART database (Simple Modular Architecture Research Tool, EMBL, Heidelberg, DE) of HMMs as described in Schultz et al. (1998), Proc. Natl. Acad. Sci. USA 95:5857 and Schultz et al. (2000) Nucl. Acids Res 28:231. The SMART database contains domains identified by profiling with the hidden Markov models of the HMMer2 search program (R. Durbin et al. (1998) Biological sequence analysis: probabilistic models of proteins and nucleic acids. Cambridge University Press). The database also is annotated and monitored. The Propom protein domain database consists of an automatic compilation of homologous domains (Corpet et al. (1999), Nucl. Acids Res. 27:263-267). Current versions of Propom are built using recursive PSI-BLAST searches (Altschul et al. (1997) Nucleic Acids Res. 25:3389-3402; Gouzy et al. (1999) Computers and Chemistry 23:333-340.) of the SWISS-PROT 38 and TREMBL protein databases. The database automatically generates a consensus sequence for each domain. Prosite lists the Kunitz domain as a motif and identifies proteins that include a Kunitz domain. See, e.g., Falquet et al. Nucleic Acids Res. 30:235-238 (2002).

Useful Kunitz domains for selecting protease inhibitors can include Kunitz domains that have a framework region with a particular number of lysine residues. In one implementation, frameworks with four lysine residues are useful and can be modified, e.g., by attachment of PEG moieties of average molecular weight between 3-8 kDa, e.g., about 5 kDa. For example, the ITI framework has four lysines. In another implementation, frameworks with three lysines are useful and can be modified e.g., by attachment of PEG moieties of average molecular weight between 4-10 kDa, e.g., about 5 kDa or 7 kDa. LACI is one such framework. Frameworks can also be altered to include fewer or additional lysines, for example, to reduce the number of lysines that are within five, four, or three residues of a binding loop, or to introduce a sufficient number of lysines that the protein can be modified with small PEG moieties (e.g., between 3-8 kDa PEG moieties) to increase the size of the protein and stability of the protein in vivo.

Kunitz domains interact with target protease using, primarily, amino acids in two loop regions (“binding loops”). The first loop region is between about residues corresponding to amino acids 11-21 of BPTI. The second loop region is between about residues corresponding to amino acids 31-42 of BPTI. An exemplary library of Kunitz domains varies one or more amino acid positions in the first and/or second loop regions. Particularly useful positions to vary include: positions 13, 16, 17, 18, 19, 31, 32, 34, and 39 with respect to the sequence of BPTI. At least some of these positions are expected to be in close contact with the target protease.

The “framework region” of a Kunitz domain is defined as those residues that are a part of the Kunitz domain, but specifically excluding residues in the first and second binding loops regions, e.g., about residues corresponding to amino acids 11-21 of BPTI and 31-42 of BPTI. The framework region can be derived from a human Kunitz domain, e.g., LACI. Exemplary frameworks can include at least one, two, or three lysines. In one embodiment, the lysines are present at positions corresponding to the positions found in the framework of LACI, or within at least three, two, or one amino acid from such a position.

Conversely, residues that are not at these particular positions or which are not in the loop regions may tolerate a wider range of amino acid substitutions (e.g., conservative and/or non-conservative substitutions) than these amino acid positions.

Exemplary methods for screening and isolating Kunitz domains with a particular specificity include those described in: US 2004-0209243, U.S. Pat. No. 5,223,409, and U.S. Pat. No. 6,423,498. Proteins that include Kunitz domains can be produced using recombinant techniques in bacteria (e.g., E. coli), yeast (e.g., Saccharomyces or Pichia), insect cells, mammalian cells, or transgenic animals (e.g., for secretion into milk).

### Plasmin Inhibitors-Antibodies

One class of plasmin inhibitors includes antibodies. Exemplary antibodies bind specifically to plasmin. An antibody can inhibit plasmin in a number of ways. For example, it can contact one or more residues of the active site, sterically hinder or obstruct access to the active site, prevent maturation of plasmin, or destabilize a conformation required for catalytic activity.

As used herein, the term “antibody” refers to a protein that includes at least one immunoglobulin variable domain or immunoglobulin variable domain sequence. For example, an antibody can include a heavy (H) chain variable region (abbreviated herein as VH), and a light (L) chain variable region (abbreviated herein as VL). In another example, an antibody includes two heavy (H) chain variable regions and two light (L) chain variable regions. The term “antibody” encompasses antigen-binding fragments of antibodies (e.g., single chain antibodies, Fab fragments, F(ab′)₂, a Fd fragment, a Fv fragments, and dAb fragments) as well as complete antibodies.

The VH and VL regions can be further subdivided into regions of hypervariability, termed “complementarity determining regions” (“CDR”), interspersed with regions that are more conserved, termed “framework regions” (FR). The extent of the framework region and CDRs has been precisely defined (see, Kabat, E. A., et al. (1991) Sequences of Proteins of Immunological Interest, Fifth Edition, U.S. Department of Health and Human Services, NIH Publication No. 91-3242, and Chothia, C. et al. (1987) J. Mol. Biol. 196:901-917). Kabat definitions are used herein. Each VH and VL is typically composed of three CDRs and four FRs, arranged from amino-terminus to carboxy-terminus in the following order: FR1, CDR1, FR2, CDR2, FR3, CDR3, FR4.

An “immunoglobulin domain” refers to a domain from the variable or constant domain of immunoglobulin molecules. Immunoglobulin domains typically contain two β-sheets formed of about seven β-strands, and a conserved disulphide bond (see, e.g., A. F. Williams and A. N. Barclay 1988 Ann. Rev Immunol. 6:381-405).

As used herein, an “immunoglobulin variable domain sequence” refers to an amino acid sequence which can form the structure of an immunoglobulin variable domain. For example, the sequence may include all or part of the amino acid sequence of a naturally-occurring variable domain. For example, the sequence may omit one, two or more N- or C-terminal amino acids, internal amino acids, may include one or more insertions or additional terminal amino acids, or may include other alterations. In one embodiment, a polypeptide that includes immunoglobulin variable domain sequence can associate with another immunoglobulin variable domain sequence to form a target binding structure (or “antigen binding site”), e.g., a structure that preferentially interacts with an activated integrin structure or a mimic of an activated integrin structure, e.g., relative to an non-activated structure.

The VH or VL chain of the antibody can further include all or part of a heavy or light chain constant region, to thereby form a heavy or light immunoglobulin chain, respectively. In one embodiment, the antibody is a tetramer of two heavy immunoglobulin chains and two light immunoglobulin chains, wherein the heavy and light immunoglobulin chains are inter-connected by, e.g., disulfide bonds. The heavy chain constant region includes three domains, CH1, CH2 and CH3. The light chain constant region includes a CL domain. The variable region of the heavy and light chains contains a binding domain that interacts with an antigen. The constant regions of the antibodies typically mediate the binding of the antibody to host tissues or factors, including various cells of the immune system (e.g., effector cells) and the first component (Clq) of the classical complement system. The term “antibody” includes intact immunoglobulins of types IgA, IgG, IgE, IgD, IgM (as well as subtypes thereof). The light chains of the immunoglobulin may be of types kappa or lambda. In one embodiment, the antibody is glycosylated. An antibody can be functional or non-functional for antibody-dependent cytotoxicity and/or complement-mediated cytotoxicity.

One or more regions of an antibody can be human or effectively human. For example, one or more of the variable regions can be human or effectively human. For example, one or more of the CDRs can be human, e.g., HC CDR1, HC CDR2, HC CDR3, LC CDR1, LC CDR2, and LC CDR3. Each of the light chain CDRs can be human. HC CDR3 can be human. One or more of the framework regions can be human, e.g., FR1, FR2, FR3, and FR4 of the HC or LC. In one embodiment, all the framework regions are human, e.g., derived from a human somatic cell, e.g., a hematopoietic cell that produces immunoglobulins or a non-hematopoietic cell. In one embodiment, the human sequences are germline sequences, e.g., encoded by a germline nucleic acid. One or more of the constant regions can be human or effectively human. In another embodiment, at least 70, 75, 80, 85, 90, 92, 95, or 98% of, or the entire antibody can be human or effectively human. An “effectively human” immunoglobulin variable region is an immunoglobulin variable region that includes a sufficient number of human framework amino acid positions such that the immunoglobulin variable region does not elicit an immunogenic response in a normal human. An “effectively human” antibody is an antibody that includes a sufficient number of human amino acid positions such that the antibody does not elicit an immunogenic response in a normal human.

All or part of an antibody can be encoded by an immunoglobulin gene or a segment thereof. Exemplary human immunoglobulin genes include the kappa, lambda, alpha (IgA1 and IgA2), gamma (IgG1, IgG2, IgG3, IgG4), delta, epsilon and mu constant region genes, as well as the myriad immunoglobulin variable region genes. Full-length immunoglobulin “light chains” (about 25 Kd or 214 amino acids) are encoded by a variable region gene at the NH2-terminus (about 110 amino acids) and a kappa or lambda constant region gene at the COOH-terminus. Full-length immunoglobulin “heavy chains” (about 50 Kd or 446 amino acids), are similarly encoded by a variable region gene (about 116 amino acids) and one of the other aforementioned constant region genes, e.g., gamma (encoding about 330 amino acids).

One exemplary method for identifying antibodies that bind to and inhibit plasmin includes immunizing a non-human animal with plasmin or a fragment thereof. Even small peptides can be used as immunogens. In one embodiment, a mutated plasmin, which has reduced, or no catalytic activity is used as immunogen. Spleen cells can be isolated from the immunized animal and used to produce hybridoma cells using standard methods. In one embodiment, the non-human animal includes one or more human immunoglobulin genes.

Another exemplary method for identifying proteins that bind to and inhibit plasmin includes: providing a library of proteins and selecting from the library one or more proteins that bind to a plasmin or a fragment thereof. The selection can be performed in a number of ways. For example, the library can be provided in the format of a display library or a protein array. Prior to selecting, the library can be pre-screened (e.g., depleted) to remove members that interact with a non-target molecule, e.g., protease other than a plasmin or a plasmin in which the active site is inaccessible, e.g., bound by an inhibitor, e.g., aprotinin.

Antibody libraries, e.g., antibody display libraries, can be constructed by a number of processes (see, e.g., de Haard et al. (1999) J. Biol. Chem. 274:18218-30; Hoogenboom et al. (1998) Immunotechnology 4:1-20. and Hoogenboom et al. (2000) Immunol Today 21:371-8). Further, elements of each process can be combined with those of other processes. The processes can be used such that variation is introduced into a single immunoglobulin domain (e.g., VH or VL) or into multiple immunoglobulin domains (e.g., VH and VL). The variation can be introduced into an immunoglobulin variable domain, e.g., in the region of one or more of CDR1, CDR2, CDR3, FR1, FR2, FR3, and FR4, referring to such regions of either and both of heavy and light chain variable domains. In one embodiment, variation is introduced into all three CDRs of a given variable domain. In another preferred embodiment, the variation is introduced into CDR1 and CDR2, e.g., of a heavy chain variable domain. Any combination is feasible.

In an exemplary system for recombinant expression of an antibody (e.g., a full length antibody or an antigen-binding portion thereof), a recombinant expression vector encoding both the antibody heavy chain and the antibody light chain is introduced into dhfr-CHO cells by calcium phosphate-mediated transfection. Within the recombinant expression vector, the antibody heavy and light chain genes are each operatively linked to enhancer/promoter regulatory elements (e.g., derived from SV40, CMV, adenovirus and the like, such as a CMV enhancer/AdMLP promoter regulatory element or an SV40 enhancer/AdMLP promoter regulatory element) to drive high levels of transcription of the genes. The recombinant expression vector also carries a DHFR gene, which allows for selection of CHO cells that have been transfected with the vector using methotrexate selection/amplification. The selected transformant host cells are cultured to allow for expression of the antibody heavy and light chains and intact antibody is recovered from the culture medium. Standard molecular biology techniques are used to prepare the recombinant expression vector, transfect the host cells, select for transformants, culture the host cells, and recover the antibody from the culture medium. For example, some antibodies can be isolated by affinity chromatography with a Protein A or Protein G. Antibodies can also be produced by a transgenic animal.

### Plasmin Inhibitors-Peptides

The binding ligand can include a peptide of 32 amino acids or less that independently binds to a target molecule. Some such peptides can include one or more disulfide bonds. Other peptides, so-called “linear peptides,” are devoid of cysteines. In one embodiment, the peptides are artificial, i.e., not present in nature or not present in a protein encoded by one or more genomes of interest, e.g., the human genome. Synthetic peptides may have little or no structure in solution (e.g., unstructured), heterogeneous structures (e.g., alternative conformations or “loosely structured), or a singular native structure (e.g., cooperatively folded). Some synthetic peptides adopt a particular structure when bound to a target molecule. Some exemplary synthetic peptides are so-called “cyclic peptides” that have at least a disulfide bond and, for example, a loop of about 4 to 12 non-cysteine residues. Exemplary peptides are less than 28, 24, 20, or 18 amino acids in length.

Peptide sequences that independently bind plasmin can be identified by any of a variety of methods. For example, they can be selected from a display library or an array of peptides. After identification, such peptides can be produced synthetically or by recombinant means. The sequences can be incorporated (e.g., inserted, appended, or attached) into longer sequences.

Exemplary phage libraries can be screened to find at least some of the peptide ligands described herein. Each library can display a short, variegated exogenous peptide on the surface of M13 phage. The peptide display of five of the libraries can be based on a parental domain having a segment of 4, 5, 6, 7, 8, 10, 11, or 12 amino acids, respectively, flanked by cysteine residues. The pairs of cysteines are believed to form stable disulfide bonds, yielding a cyclic display peptide. The cyclic peptides can be displayed at the amino terminus of protein III on the surface of the phage. A phage library with a 20 amino acid linear display can also be screened.

The techniques discussed in Kay et al., Phage Display of Peptides and Proteins: A Laboratory Manual (Academic Press, Inc., San Diego 1996) and U.S. Pat. No. 5,223,409 are useful for preparing a library of potential binders corresponding to the selected parental template. The libraries can be prepared according to such techniques, and screened, e.g., for peptides that bind to and inhibit plasmin.

In addition, phage libraries or selected populations from phage libraries can be counter-selected, e.g., using plasmin that is inactivated, e.g., by binding of aprotinin or another plasmin inhibitor. Such procedures can be used to discard peptides that do not contact the active site.

Peptides can also be synthesized using alternative backbones, e.g., a peptoid backbone, e.g., to produce a compound that has increased protease resistance. In particular, this method can be used to make a compound that binds to and inhibits plasmin and which is not itself effectively cleaved by plasmin.

Still other inhibitors of plasmin include small molecules (e.g., molecules smaller than 700 Daltons or molecules that include fewer than four peptides bonds). Additional exemplary plasmin inhibitors include: alpha2-plasmin inhibitor and alpha2-macroglobulin,

### Pharmaceutical Compositions

Also featured is a composition, e.g., a pharmaceutically acceptable composition, that includes a compound that includes a plasmin inhibitor, e.g., a protein that includes a Kunitz domain that inhibits plasmin, e.g., DX-1000. In one embodiment, the protein is modified, e.g., with a polymer such as PEG. Pharmaceutical compositions encompass compounds (e.g., labeled compounds) for diagnostic (e.g., in vivo imaging) use as well as compounds for therapeutic or prophylactic use.

A pharmaceutically acceptable carrier includes any and all solvents, dispersion media, coatings, anti-bacterial and anti-fungal agents, isotonic and absorption delaying agents, and the like that are physiologically compatible. In one embodiment, the carrier is other than water. Preferably, the carrier is suitable for intravenous, intramuscular, subcutaneous, parenteral, spinal or epidermal administration (e.g., by injection or infusion). Depending on the route of administration, the active compound may be coated in a material to protect the compound from the action of acids and other natural conditions that may inactivate the compound.

A pharmaceutically acceptable salt is a salt that retains the desired biological activity of the parent compound and does not impart any undesired toxicological effects (see, e.g., Berge, S. M., et al. (1977) J. Pharm. Sci. 66:1-19). Examples of such salts include acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like.

The compositions may be in a variety of forms. These include, for example, liquid, semi-solid and solid dosage forms, such as liquid solutions (e.g., injectable and infusible solutions), dispersions or suspensions, tablets, pills, powders, liposomes and suppositories. The form can depend on the intended mode of administration and therapeutic application. Typical compositions are in the form of injectable or infusible solutions, such as compositions similar to those used for administration of humans with antibodies. Administration can be parenteral. Examples of parenteral administration include intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion. In one embodiment, the compound is administered by intravenous infusion or injection. In another embodiment, the compound is administered by intramuscular or subcutaneous injection.

Pharmaceutical compositions typically are sterile and stable under the conditions of manufacture and storage. A pharmaceutical composition can also be tested to insure it meets regulatory and industry standards for administration. For example, endotoxin levels in the preparation can be tested using the Limulus amebocyte lysate assay (e.g., using the kit from Bio Whittaker lot #7L3790, sensitivity 0.125 EU/mL) according to the USP 24/NF 19 methods. Sterility of pharmaceutical compositions can be determined using thioglycollate medium according to the USP 24/NF 19 methods. For example, the preparation is used to inoculate the thioglycollate medium and incubated at 35° C. for 14 or more days. The medium is inspected periodically to detect growth of a microorganism.

The composition can be formulated as a solution, microemulsion, dispersion, liposome, or other ordered structure suitable to high drug concentration. Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

The plasmin inhibitors described herein can be administered by a variety of methods. For many applications, the route/mode of administration is intravenous injection, subcutaneous injection, or infusion. For example, for therapeutic applications, the compound can be administered by intravenous infusion, e.g., at a rate of less than 30, 20, 10, 5, or 1 mg/min to reach a dose of about 1 to 100 mg/m² or 7 to 25 mg/m². The route and/or mode of administration can vary depending upon the desired results.

In certain embodiments, the plasmin inhibitor may be prepared with a carrier that will protect the inhibitor against rapid release, such as a controlled release formulation, including implants, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known. See, e.g., Sustained and Controlled Release Drug Delivery Systems, J. R. Robinson, ed., Marcel Dekker, Inc., New York, 1978. Pharmaceutical formulation is a well-established art, and is further described in Gennaro (ed.), Remington: The Science and Practice of Pharmacy, 20^{th} ed., Lippincott, Williams & Wilkins (2000) (ISBN: 0683306472); Ansel et al., Pharmaceutical Dosage Forms and Drug Delivery Systems, 7^{th} Ed., Lippincott Williams & Wilkins Publishers (1999) (ISBN: 0683305727); and Kibbe (ed.), Handbook of Pharmaceutical Excipients American Pharmaceutical Association, 3^{rd} ed. (2000) (ISBN: 091733096X).

In certain embodiments, the composition may be orally administered, for example, with an inert diluent or an assimilable edible carrier. The compound (and other ingredients, if desired) may also be enclosed in a hard or soft shell gelatin capsule, compressed into tablets, or incorporated directly into the subject's diet. For oral therapeutic administration, the compound may be incorporated with excipients and used in the form of ingestible tablets, buccal tablets, troches, capsules, elixirs, suspensions, syrups, wafers, and the like. To administer a compound by other than parenteral administration, it may be necessary to coat the compound with, or co-administer the compound with, a material to prevent its inactivation.

Pharmaceutical compositions can be administered with a medical device. Exemplary medical devices include a needleless hypodermic injection device, infusion pumps, osmotic delivery systems, and so forth.

The plasmin inhibitor can be administered in order to provide an effective amount of the inhibitor, an amount able to ameliorate the disorder or to prevent further deterioration of the disorder. Dosage regimens can be adjusted to provide the optimum desired response (e.g., a therapeutic response). Dosages can be based on judgment of the attending physician and/or pharmacist, e.g., in view of individual circumstances and/or available in vivo or in vitro data. For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit forms can be used to provide unitary dosages for the subjects to be treated; each unit contains a predetermined quantity of active compound calculated to produce the desired therapeutic effect in association with the required pharmaceutical carrier. The specification for the dosage unit forms can be dictated by and directly dependent on (a) the unique characteristics of the active compound and the particular therapeutic effect to be achieved, and (b) the limitations inherent in compounding such an active compound for the treatment of sensitivity in individuals.

In certain embodiments, the compound can be formulated to ensure proper distribution in vivo. For example, the compound can be formulated using a liposome or by attachment to an appropriate moiety for delivery across the blood-brain barrier (BBB). The liposomes may comprise one or more moieties that are selectively transported into specific cells or organs, thus enhance targeted drug delivery (see, e.g., V. V. Ranade (1989) J. Clin. Pharmacol. 29:685).

The disclosure also provides a kit that includes a plasmin inhibitor, e.g., a plasmin inhibitor described herein and instructions for use, e.g., treatment, prophylactic, or diagnostic use. In one embodiment, the kit includes (a) the compound, e.g., a composition that includes the compound, and, optionally, (b) informational material. The informational material can be descriptive, instructional, marketing or other material that relates to the methods described herein and/or the use of the compound for the methods described herein. For example, the informational material describes methods for administering the compound to modulate metastatic cancer or angiogenesis-related disorder.

In one embodiment, the informational material can include instructions to administer the compound in a suitable manner, e.g., in a suitable dose, dosage form, or mode of administration (e.g., a dose, dosage form, or mode of administration described herein). In another embodiment, the informational material can include instructions for identifying a suitable subject, e.g., a human, e.g., a human having, or at risk for a disorder characterized by excessive plasmin activity. The informational material can include information about production of the compound, molecular weight of the compound, concentration, date of expiration, batch or production site information, and so forth. The informational material of the kits is not limited in its form.

The kit can further contain a least one additional reagent, such as a diagnostic or therapeutic agent, e.g., a diagnostic or therapeutic agent as described herein, and/or one or more additional agents to treat a metastatic cancer or an angiogenesis-related disorder.

### Polymers

A variety of moieties can be used to increase the molecular weight and/or half-life of a protein that includes a Kunitz domain or other protease inhibitor. In one embodiment, the moiety is a polymer, e.g., a water soluble and/or substantially non-antigenic polymer such as a homopolymer or a non-biological polymer. Substantially non-antigenic polymers include, e.g., polyalkylene oxides or polyethylene oxides. The moiety may improve stabilization and/or retention of the Kunitz domain in circulation, e.g., in blood, serum, lymph, or other tissues, e.g., by at least 1.5, 2, 5, 10, 50, 75, or 100 fold. A plurality of moieties are attached to a Kunitz domain. For example, the protein is attached to at least three moieties of the polymer. Each lysine of the protein can be attached to a moiety of the polymer. Generally, a Kunitz domain described herein can be modified as described in U.S. Ser. No. 10/931,153. Kunitz domains having sequences or conforming to motifs described in U.S. Pat. No. 6,103,499 can be modified as described herein.

Suitable polymers can vary substantially by weight. For example, it is possible to use polymers having average molecular weights ranging from about 200 Daltons to about 40 kDa, e.g., 1-20 kDa, 4-12 kDa or 3-8 kDa, e.g., about 4, 5, 6, or 7 kDa. In one embodiment, the average molecular weight of individual moieties of the polymer that are associated with the compound are less than 20, 18, 17, 15, 12, 10, 8, or 7 kDa. The final molecular weight can also depend upon the desired effective size of the conjugate, the nature (e.g. structure, such as linear or branched) of the polymer, and the degree of derivatization.

A non-limiting list of exemplary polymers include polyalkylene oxide homopolymers such as polyethylene glycol (PEG) or polypropylene glycols, polyoxyethylenated polyols, copolymers thereof and block copolymers thereof, provided that the water solubility of the block copolymers is maintained. The polymer can be a hydrophilic polyvinyl polymers, e.g. polyvinylalcohol and polyvinylpyrrolidone. Additional useful polymers include polyoxyalkylenes such as polyoxyethylene, polyoxypropylene, and block copolymers of polyoxyethylene and polyoxypropylene (Pluronics); polylactic acid; polyglycolic acid; polymethacrylates; carbomers; branched or unbranched polysaccharides which comprise the saccharide monomers D-mannose, D- and L-galactose, fucose, fructose, D-xylose, L-arabinose, D-glucuronic acid, sialic acid, D-galacturonic acid, D-mannuronic acid (e.g. polymannuronic acid, or alginic acid), D-glucosamine, D-galactosamine, D-glucose and neuraminic acid including homopolysaccharides and heteropolysaccharides such as lactose, cellulose, amylopectin, starch, hydroxyethyl starch, amylose, dextrane sulfate, dextran, dextrins, glycogen, or the polysaccharide subunit of acid mucopolysaccharides, e.g. hyaluronic acid; polymers of sugar alcohols such as polysorbitol and polymannitol; heparin or heparon. In some embodiments, the polymer includes a variety of different copolymer blocks.

The protein that includes a Kunitz domain can be physically associated with the polymer in a variety of ways. Typically, the protein is covalently linked to the polymer at a plurality of sites. For example, the protein is conjugated to the polymer at a plurality of primary amines, e.g., all accessible primary amines or all primary amines. Other compounds can also be attached to the same polymer, e.g., a cytotoxin, a label, or another targeting agent, e.g., another ligand that binds to the same target as the Kunitz domain or a ligand that binds to another target, e.g., a an unrelated ligand. Other compounds may also be attached to the protein.

In one embodiment, the polymer is water soluble prior to conjugation to the protein (although need not be). Generally, after conjugation to the protein, the product is water soluble, e.g., exhibits a water solubility of at least about 0.01 mg/ml, and more preferably at least about 0.1 mg/ml, and still more preferably at least about 1 mg/ml. In addition, the polymer should not be highly immunogenic in the conjugate form, nor should it possess viscosity that is incompatible with intravenous infusion or injection if the conjugate is intended to be administered by such routes.

In one embodiment, the polymer contains only a single group which is reactive. This helps to avoid conjugation of one polymer to multiple protein molecules. Mono-activated, alkoxy-terminated polyalkylene oxides (PAO's), e.g., monomethoxy-terminated polyethylene glycols (mPEG's); C₁₋₄ alkyl-terminated polymers; and bis-activated polyethylene oxides (glycols) can be used for conjugation to the protein. See, e.g., U.S. Pat. No. 5,951,974.

In its most common form, poly(ethylene glycol), PEG, is a linear or branched polyether terminated with hydroxyl groups. Linear PEG can have the following general structure:

HO—(CH₂CH₂O)ₙ—CH₂CH₂—OH

PEG can be synthesized by anionic ring opening polymerization of ethylene oxide initiated by nucleophilic attack of a hydroxide ion on the epoxide ring. Particularly useful for protein modification is monomethoxy PEG, mPEG, having the general structure:

CH₃O—(CH₂CH₂O)ₙ—CH₂CH₂—OH

For further descriptions, see, e.g., Roberts et al. (2002) Advanced Drug Delivery Reviews 54:459-476. In one embodiment, the polymer units used for conjugation are mono-disperse or otherwise highly homogenous, e.g., present in a preparation in which 95% or all molecules are with 7, 5, 4, 3, 2, or 1 kDa of one another. In another embodiment, the polymer units are poly-disperse.

It is possible to select reaction conditions that reduce cross-linking between polymer units or conjugation to multiple proteins and to purify the reaction products through gel filtration or ion exchange chromatography to recover substantially homogenous derivatives, e.g., derivatives that include only a single Kunitz domain protein. In other embodiments, the polymer contains two or more reactive groups for the purpose of linking multiple proteins (e.g., multiple units of the Kunitz domain protein) to the polymer. Again, gel filtration or ion exchange chromatography can be used to recover the desired derivative in substantially homogeneous form.

The protein that includes a Kunitz domain is generally attached to a plurality of PEG molecules. For example, to form a compound that is larger than 20 or 30 kDa, a Kunitz domain (about 7 kDa) can be attached to at least three 8 kDa molecules of PEG. Other combinations are possible, e.g., at least two, four, or five molecules of PEG. The molecular weight of the PEG molecules can be selected so that the final molecular weight of the compound is equal to or larger than a desired molecular weight (e.g., between 17-35, or 20-25, or 27-33 kDa). The plurality of PEG molecules can be attached to any region of the Kunitz domain, preferably at least 5, 10, or 15 Angstroms from a region that interacts with a target, or at least 2, 3, or 4 residues from an amino acid that interacts with a target. The PEG molecules can be attached, e.g., to lysine residues or a combination of lysine residues and the N-terminus.

A covalent bond can be used to attach a protein (e.g., a protein that includes a Kunitz domain) to a polymer, for example, conjugation to the N-terminal amino group and epsilon amino groups found on lysine residues, as well as other amino, imino, carboxyl, sulfhydryl, hydroxyl or other hydrophilic groups. The polymer may be covalently bonded directly to the protein without the use of a multifunctional (ordinarily bifunctional) crosslinking agent. Covalent binding to amino groups can be accomplished by known chemistries based upon cyanuric chloride, carbonyl diimidazole, aldehyde reactive groups (PEG alkoxide plus diethyl acetyl of bromoacetaldehyde; PEG plus DMSO and acetic anhydride, or PEG chloride plus the phenoxide of 4-hydroxybenzaldehyde, activated succinimidyl esters, activated dithiocarbonate PEG, 2,4,5-trichlorophenylcloroformate or P-nitrophenylcloroformate activated PEG.) Carboxyl groups can be derivatized by coupling PEG-primary amine using carbodiimide. Sulfhydryl groups can be derivatized by coupling to maleimido-substituted PEG (see, e.g., WO 97/10847) or PEG-maleimide (e.g., commercially available from Shearwater Polymers, Inc., Huntsville, Ala.). Alternatively, free amino groups on the protein (e.g. epsilon amino groups on lysine residues) can be thiolated with 2-imino-thiolane (Traut's reagent) and then coupled to maleimide-containing derivatives of PEG, e.g., as described in Pedley et al., Br. J. Cancer, 70: 1126-1130 (1994).

Functionalized PEG polymers that can be attached to a protein that includes Kunitz domain include polymers that are commercially available, e.g., from Shearwater Polymers, Inc. (Huntsville, Ala.). Such PEG derivatives include, e.g., amino-PEG, PEG amino acid esters, PEG-hydrazide, PEG-thiol, PEG-succinate, carboxymethylated PEG, PEG-propionic acid, PEG amino acids, PEG succinimidyl succinate, PEG succinimidyl propionate, succinimidyl ester of carboxymethylated PEG, succinimidyl carbonate of PEG, and others. The reaction conditions for coupling these PEG derivatives may vary depending on the protein, the desired degree of PEGylation, and the PEG derivative utilized. Some factors involved in the choice of PEG derivatives include: the desired point of attachment (such as lysine or cysteine R-groups), hydrolytic stability and reactivity of the derivatives, stability, toxicity and antigenicity of the linkage, suitability for analysis, etc.

The conjugates of a protein that includes a Kunitz domain and a polymer can be separated from the unreacted starting materials using chromatographic methods, e.g., by gel filtration or ion exchange chromatography, e.g., HPLC. Heterologous species of the conjugates are purified from one another in the same fashion. Resolution of different species (e.g. containing one or two PEG residues) is also possible due to the difference in the ionic properties of the unreacted amino acids. See, e.g., WO 96/34015.

In one embodiment, non-protein moieties (e.g., a polymer described herein) are attached to each available primary amine on the Kunitz domain, e.g., the N-terminal primary amine and any solvent-accessible primary amines, e.g., accessible primary amines of lysine side chains in the Kunitz domain. For example, all possible primary amines are conjugated to one of the non-protein moieties. The Kunitz domain may have at least one, two, three, or four lysines. For example, the Kunitz domain may have only one, two, three, four, or five lysines. In one embodiment, the protein has an N-terminal primary amine. In another embodiment, the protein does not include an N-terminal primary amine (e.g., the protein can be chemically modified, e.g., with a non-polymeric compound, at its N-terminal primary amine so that the protein does not include a primary amine at that position).

A non-protein moiety (e.g., a polymer) can be attached at two or more of the primary amines in the protein. For example, all lysines or all lysines that have a solvent accessible primary amine are attached to a non-protein moiety. Preferably, the Kunitz domain does not include a lysine within one of its binding loops, e.g., about residues corresponding to amino acids 11-21 of BPTI and 31-42 of BPTI. Lysines within such binding loops can be replaced, e.g., with arginine residues. For example, the protein is attached to at least three of molecules of the polymer. Each lysine of the protein, or one, two, three or more of the lysines can be attached to a molecule of the polymer.

Unless otherwise stated, when it is said that a primary amine, e.g., that of a particular lysine or at the N terminus, is modified or has a non-protein moiety attached thereto, it is understood that the specified primary amine position on every molecule in a preparation may not be so modified. The preparations need not be perfectly homogeneous. Homogeneity is desirable in some embodiments but it need not be absolute. In preferred embodiments, at least 60, 70, 80, 90, 95, 97, 98, 99, or 100% of a primary amine which is designated as modified will have a non-protein moiety attached thereto. Other embodiments however, include preparations that contains a mixture of species in which most of the molecules, e.g., at least 60, 70, 80, 90, 95, 97, 98, 99, or 100% are PEGylated at two or more sites but the sites (and in some cases the number of sites modified) on molecules in the preparation will vary. E.g., some molecules will have lysines A, B, and D modified while other molecules will have the amino terminus and lysines A, B, C, and D modified. Preparations such as these can be administered to a subject, e.g., according to a treatment or therapeutic method described herein.

In one embodiment, the non-protein moiety includes a hydrophilic polymer, e.g., a substantially homogeneous polymer. The polymer can be branched or unbranched. For example, the moiety of polymer has a molecular weight (e.g., an average molecular weight of the moieties added to the compound) that is less than 20, 18, 15, 12, 10, 8, 7, or 6 or at least 1.5, 2, 2.5, 3, 5, 6, 10 kDa, e.g., about 5 kDa. In one embodiment, the sum of the molecular weight of the PEG moieties on the compound is at least 15, 20, 25, 30, or 35, and/or less than 60, 50, 40, 35, 30, 25, or 23 kDa.

In one embodiment, the polymer is a polyalkylene oxide. For example, at least 20, 30, 50, 70, 80, 90, or 95% of the copolymer blocks of the polymer are ethylene glycol. In one embodiment, the polymer is polyethylene glycol.

In one embodiment, the compound has the following structure: P—X⁰—[(CR′R″)ₙ—X¹]ₐ—(CH₂)ₘ—X²—R^{t} wherein P is the protein, each of R′ and R″ is, independently, H, or C₁-C₁₂ alkyl; X⁰ is O, N—R¹, S, or absent, wherein R¹ is H, C₁-C₁₂ alkyl or aryl, X¹ is O, N—R², S, wherein R² is H, alkyl or aryl, X² is O, N—R³, S, or absent, wherein R³ is H, alkyl or aryl, each n is between 1 and 5, e.g., 2, a is at least 4, m is between 0 and 5, and R^{t} is H, C₁-C₁₂ alkyl or aryl. R′ and R″ can be H. In one embodiment, R′ or R″ is independently, H, or C1-C4, C1-C6, or C1-C10 alkyl.

In one embodiment, the compound has the following structure: P—X⁰—[CH₂CH₂O]ₐ—(CH₂)ₘ—X²—R^{t} wherein P is the protein, a is at least 4, m is between 0 and 5, X² is O, N—R′, S, or absent, wherein R¹ is H, alkyl or aryl, X⁰ is O, N—R², S, or absent, wherein R² is H, alkyl or aryl, and R^{t} is H, C₁-C₁₂ alkyl or aryl. For example, X² is O, and R^{t} is CH3. (The use of mPEG is preferred.) In one embodiment, the Kunitz domain protein is less than 14, 8, or 7 kDa in molecular weight. In one embodiment, the Kunitz domain protein includes only one Kunitz domain. Generally, the compound includes only one Kunitz domain, but in some embodiments, may include more than one.

In one embodiment, the Kunitz domain includes the amino acid sequence of DX-1000 or an amino acid sequence that differs by at least one, but fewer than six, five, four, three, or two amino acid differences (e.g., substitutions, insertions, or deletions) from the amino acid sequence of DX-1000. Typically, the Kunitz domain does not naturally occur in humans. The Kunitz domain may include an amino acid sequence that differs by fewer than ten, seven, or four amino acids from a human Kunitz domain.

In one embodiment, the Kᵢ of the compound is within a factor of 0.5 to 1.5, 0.8 to 1.2, 0.3 to 3.0, 0.1 to 10.0, or 0.02 to 50.0 of the Kᵢ of the unmodified protein for elastase. For example, the Kᵢ for hNE can be less than 100, 50, 18, 12, 10, or 9 μM.

In one embodiment, the compound has a circulatory half life of the longest-lived component (“longest phase circulatory half life”) in a rabbit or mouse model that is at least 1.5, 2, 4, or 8 fold greater than a substantially identical compound that does not include the polymer. The compound can have a longest phase circulatory half life in a rabbit or mouse model that has an amplitude at least 1.5, 2, 2.5, or 4 fold greater than a substantially identical compound that does not include the non-protein moiety. The compound can have an alpha-phase circulatory half life in a rabbit or mouse model that has an amplitude at least 20, 30, 40, or 50% smaller than a substantially identical compound that does not include the non-protein moiety. For example, the compound has a longest phase circulatory half life with an amplitude of at least 40, 45, 46, 50, 53, 54, 60, or 65%. In one embodiment, the compound has a beta phase circulatory half life in a mouse or rabbit model of at least 2, 3, 4, 5, 6, or 7 hours. In one embodiment, the compound has a longest phase circulatory half life in a 70 kg human of at least 6 hours, 12 hours, 24 hours, 2 days, 5 days, 7 days, or 10 days.

### Serum Albumin Fusion

A plasmin inhibitor such as a protein comprising a Kunitz domain, e.g., DX-1000 or other protein described herein, can be fused to a carrier protein, e.g., serum albumin, or a fragment thereof, to stabilize, prolong or extend the in vivo half-life, therapeutic activity or shelf life of the plasmin inhibitor portion of the albumin fusion protein compared to the in vivo half-life, therapeutic activity, or shelf-life of the plasmin inhibitor in the non-fusion state (see, e.g., US-2004-0171794).

As used herein, “albumin” refers collectively to albumin protein or amino acid sequence, or an albumin fragment or variant, having one or more functional activities (e.g., biological activities) of albumin. One example of an albumin is human albumin or fragments thereof (see, e.g., EP 201 239, EP 322 094, WO 97/24445, WO95/23857).

In particular, the albumin fusion proteins of the claimed methods may include naturally occurring polymorphic variants of human albumin and fragments of human albumin, for example those fragments disclosed in EP 322 094 (namely HA (Pn), where n is 369 to 419). The albumin may be derived from any vertebrate, especially any mammal, for example human, cow, sheep, or pig. Non-mammalian albumins include, but are not limited to, hen and salmon. The albumin portion of the albumin fusion protein may be from a different animal than the plasmin inhibitor portion.

The albumin portion of an albumin fusion protein of the claimed methods may comprise at least one subdomain or domain of albumin or conservative modifications thereof. If the fusion is based on subdomains, some or all of the adjacent linker may optionally be used to link to the plasmin inhibitor.

The albumin fusion protein may comprise albumin as the N-terminal portion, and the plasmin inhibitor as the C-terminal portion. Alternatively, an albumin fusion protein can comprise albumin as the C-terminal portion, and the plasmin inhibitor as the N-terminal portion.

The albumin fusion protein may comprise the plasmin inhibitor fused to both the N-terminus and the C-terminus of albumin. In one embodiment, the plasmin inhibitors fused at the N- and C-termini are the same plasmin inhibitors. In another embodiment, the plasmin inhibitors fused at the N- and C-termini are different plasmin inhibitors.

### Treating Cancers

A plasmin inhibitor, e.g., a protein that includes a Kunitz domain that inhibits plasmin, e.g., DX-1000, can be used to treat a variety of cancers, particularly metastatic cancers or cancers at risk for progressing to a metastatic stage or angiogenesis-dependent cancers or lymphangiogenesis-related cancers. A cancer refers to one or more cells that has a loss of responsiveness to normal growth controls, and typically proliferates with reduced regulation relative to a corresponding normal cell.

Examples of cancerous disorders include, but are not limited to, solid tumors, soft tissue tumors, and metastatic lesions. Examples of solid tumors include malignancies, e.g., sarcomas, adenocarcinomas, and carcinomas, of the various organ systems, such as those affecting lung, breast, lymphoid, gastrointestinal (e.g., colon), and genitourinary tract (e.g., renal, urothelial cells), pharynx, prostate, ovary as well as adenocarcinomas which include malignancies such as most colon cancers, rectal cancer, renal-cell carcinoma, liver cancer, non-small cell carcinoma of the lung, cancer of the small intestine and so forth. Metastatic lesions of the aforementioned cancers can also be treated or prevented using the methods described herein. Some cancers can express high levels of urokinase, which can lead to excessive generation of plasmin. Such cancers expressing high levels of urokinase can also be treated or prevented using the methods described herein.

For example, a plasmin inhibitor can be used to treat malignancies of the various organ systems, such as those affecting lung, breast, lymphoid, gastrointestinal (e.g., colon), and genitourinary tract, prostate, ovary, pharynx, as well as adenocarcinomas which include malignancies such as most colon cancers, renal-cell carcinoma, prostate cancer and/or testicular tumors, non-small cell carcinoma of the lung, cancer of the small intestine and cancer of the esophagus. Exemplary solid tumors that can be treated include: fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, non-small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, and retinoblastoma.

Fibrosarcoma is a soft-tissue tumor composed of fascicles of spindled fibroblast-like cells. Fibrosarcomas of the bone are often composed of a malignant spindle cell stroma that, in many instances, produce abundant collagen.

A carcinoma is a malignancy of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. Exemplary carcinomas include those forming from tissue of the cervix, lung, prostate, breast, head and neck, colon and ovary. The term also includes carcinosarcomas, e.g., which include malignant tumors composed of carcinomatous and sarcomatous tissues. An adenocarcinoma is a carcinoma derived from glandular tissue or in which the tumor cells form recognizable glandular structures.

In one embodiment, the treatment includes administering (i) a plasmin inhibitor, e.g., a protein that includes a Kunitz domain, e.g., DX-1000, and (ii) a second therapeutic agent, e.g., an anti-cancer agent. Exemplary anti-cancer agents include, e.g., anti-microtubule agents, topoisomerase inhibitors, anti-metabolites, mitotic inhibitors, alkylating agents, intercalating agents, agents capable of interfering with a signal transduction pathway, agents that promote apoptosis, radiation, and antibodies against other tumor-associated antigens (including naked antibodies, immunotoxins and radioconjugates). Examples of the particular classes of anti-cancer agents are provided in detail as follows: anti-tubulin/anti-microtubule, e.g., paclitaxel, vincristine, vinblastine, vindesine, vinorelbin, taxotere; topoisomerase I inhibitors, e.g., topotecan, camptothecin, doxorubicin, etoposide, mitoxantrone, daunorubicin, idarubicin, teniposide, amsacrine, epirubicin, merbarone, piroxantrone hydrochloride; antimetabolites, e.g., 5-fluorouracil (5-FU), methotrexate, 6-mercaptopurine, 6-thioguanine, fludarabine phosphate, cytarabine/Ara-C, trimetrexate, gemcitabine, acivicin, alanosine, pyrazofurin, N-Phosphoracetyl-L-Asparate=PALA, pentostatin, 5-azacitidine, 5-Aza 2′-deoxycytidine, ara-A, cladribine, 5-fluorouridine, FUDR, tiazofurin, N-[54N-(3,4-dihydro-2-methyl-4-oxoquinazolin-6-ylmethyl)-N-methylamino-1-2-thenoyl]-L-glutamic acid; alkylating agents, e.g., cisplatin, carboplatin, mitomycin C, BCNU=Carmustine, melphalan, thiotepa, busulfan, chlorambucil, plicamycin, dacarbazine, ifosfamide phosphate, cyclophosphamide, nitrogen mustard, uracil mustard, pipobroman, 4-ipomeanol; agents acting via other mechanisms of action, e.g., dihydrolenperone, spiromustine, and desipeptide; biological response modifiers, e.g., to enhance anti-tumor responses, such as interferon; apoptotic agents, such as actinomycin D; and anti-hormones, for example anti-estrogens such as tamoxifen or, for example antiandrogens such as 4′-cyano-3-(4-fluorophenylsulphonyl)-2-hydroxy-2-methyl-3′-(trifluoromethyl) propionanilide.

In one embodiment, the treatment includes administering (i) a plasmin inhibitor, e.g., a protein that includes a Kunitz domain, e.g., DX-1000, and (ii) a second therapeutic agent, e.g., plasma kallikrein inhibitor, e.g., DX-88 or a protein that is at least 80, 85, 90, 95, 96, 97, 98%, or 100% identical to DX-88. The amino acid sequence of DX-88 is disclosed in US-2005-0089515.

In another aspect, the disclosure features administering a plasmin inhibitor such as a protein comprising a Kunitz domain, e.g., DX-1000, as an adjuvant therapy, e.g., to a subject. The adjuvant therapy can be a post-operative therapy that is administered to the subject after the subject has undergone surgery to remove all or part of a tumor (e.g., after surgery to treat prostate, or breast, or glioblastoma, or colorectal, or lung cancer). In one embodiment, the protein comprising a Kunitz domain is administered within 6, 12, 24, 48, or 100 hours of surgery. The protein can be administered before, during, as well as after surgery.

### Treating Prostate Cancer

Prostate cancer is characterized by cancerous cells originating from the prostate. At early stages, the cancerous cells are confined to the prostate, but, if metastatic, the cells can migrate to nearby lymph glands, the seminal vesicles, and to remote sites in the body.

The exams and tests for detecting prostate cancer may include a digital rectal exam, transrectal ultrasound, cystoscopy, a urine test to check for blood or infection, a blood test to measure PSA level, and biopsies.

Prostate cancer can be assigned to one of four stages. Stage I is cancer that cannot be felt during a rectal exam. It is found by chance when surgery is done for another reason, usually for BPH. Cancer is found only in the prostate. Stage 11 is more advanced cancer, but it has not spread outside the prostate. Stage III is cancer that has spread beyond the outer layer of the prostate. It may be found in the seminal vesicles, but it has not spread to the lymph nodes. Stage IV is characterized by one or more of the following features: cancer that has invaded the bladder, rectum, or other nearby structures (other than the seminal vesicles); cancer that has spread to lymph nodes; and cancer that has spread to other parts of the body, such as the bones. A plasmin inhibitor, e.g., a protein that includes a Kunitz domain, e.g., DX-1000, can be used to treat prostate cancer at any of these stages, particular at Stage 11, III, or IV.

Prostate cancer can also be staged using Gleason scoring of pathological samples. Scores range from 2 to 10 and indicate how likely it is that a tumor will spread. A low Gleason score means the cancer cells are similar to normal prostate cells and are less likely to spread, whereas a high Gleason score means the cancer cells are very different from normal and are more likely to spread. A plasmin inhibitor, e.g., a protein that includes a Kunitz domain, e.g., DX-1000, can be used to treat prostate cancer that is characterized by a Gleason score of three or greater, e.g., at least 5, 6, 7, or 8.

Treatment for prostate cancer may include a combination of at least two therapies, for example, administering a plasmin inhibitor, e.g., one described herein, in combination with a second therapy. Examples of a second therapy include surgery, radiation therapy, and hormonal therapy. Exemplary surgical therapies include, e.g., radical retropubic prostatectomy, radical perineal prostatectomy, and transurethral resection of the prostate (TURP). Radiation therapy can be internal or external. Internal radiation therapy (implant radiation or brachytherapy) can include implanting a radiation source (e.g., a seed or needle) in or near cancerous tissue.

In one embodiment, the treatment includes administering (i) a plasmin inhibitor, e.g., a protein that includes a Kunitz domain, e.g., DX-1000, and (ii) a hormonal therapeutic. Exemplary hormonal therapies include: luteinizing hormone-releasing hormone (LH-RH) agonists (e.g., leuprolide and goserelin), anti-androgens (e.g., flutamide, bicalutamide, and nilutamide), and agents that can prevent the adrenal glands from making testosterone (e.g., ketoconazole and aminoglutethimide).

In another aspect, the disclosure features administering a plasmin inhibitor such a protein comprising a Kunitz domain, e.g., DX-1000 or other protein described herein, as an adjuvant therapy, e.g., to a subject. The adjuvant therapy can be a post-operative therapy that is administered to the subject after the subject has undergone surgery to remove all or part of a tumor (e.g., after surgery to treat prostate, or breast, or glioblastoma, or colorectal, or lung cancer). In one embodiment, the plasmin inhibitor is administered within 6, 12, 24, 48, or 100 hours of surgery. The plasmin inhibitor can be administered before, during, as well as after surgery.

### Treating Breast Cancer

Breast cancer is a significant health problem in the United States and throughout the world. It develops as the result of a pathologic transformation of normal breast epithelium into an invasive cancer.

Breast cancer is classified in stages O-IV. Stage 0 is sometimes called noninvasive carcinoma or carcinoma in situ and includes both lobular carcinoma (LCIS) and ductal carcinoma in situ (DCIS). Stages I and II are early stages, in which the cancer has spread beyond the lobe or duct and invaded nearby tissue. Stage III is called locally advanced cancer. Here, the cancer has spread to the underarm lymph nodes or other lymph nodes near the breast. Stage 1V is metastatic cancer that has spread beyond the breast and underarm lymph nodes to other parts of the body. Breast cancer can metastasize to e.g., lymph nodes, bone, lung, brain, liver, meninges, pleura, skin, eye, and bladder. Recurrent cancer means that the disease has returned in spite of the initial treatment. The main types of breast cancer are ductal carcinoma in situ, invasive ductal carcinoma, lobular carcinoma in situ, invasive lobular carcinoma, medullary carcinoma, and Paget's disease of the nipple.

A plasmin inhibitor, e.g., a protein that includes a Kunitz domain, e.g., DX-1000 or other protein described herein, can be used to treat breast cancer, e.g., at any of the above-described stages and/or types of breast cancer. In one embodiment, treatment for breast cancer may include a combination of at least two therapies, for example, administering a plasmin inhibitor, e.g., one described herein, in combination with a second therapy. Examples of a second therapy include surgery, radiation therapy, and hormonal therapy.

In another aspect, the disclosure features administering a plasmin inhibitor such as a protein comprising a Kunitz domain, e.g., DX-1000 or other protein described herein, as an adjuvant therapy, e.g., to a subject. The adjuvant therapy can be a post-operative therapy that is administered to the subject after the subject has undergone surgery to remove all or part of a tumor (e.g., after surgery to treat breast, or prostate, or glioblastoma, or colorectal, or lung cancer). In one embodiment, a plasmin inhibitor, e.g., a protein comprising a Kunitz domain that inhibits plasmin is administered within 6, 12, 24, 48, or 100 hours of surgery. The plasmin inhibitor can be administered before, during, as well as after surgery.

### Treating Angiogenesis-Related Disorders

A plasmin inhibitor such as a protein comprising a Kunitz domain, e.g., DX-1000 or other protein described herein, can be used to inhibit (e.g., inhibit at least one activity of, reduce proliferation, migration, growth or viability) of a cell, e.g., an endothelial cell in vivo. This method includes: administering the plasmin inhibitor alone or in combination with another therapeutic, to a subject requiring such treatment.

A plasmin inhibitor such as a protein comprising a Kunitz domain, e.g., DX-1000 or other protein described herein, can be used to treat or prevent angiogenesis-related disorders, particularly angiogenesis-dependent cancers and tumors. Angiogenesis-related disorders include, but are not limited to, solid tumors; tumor metastasis; benign tumors (e.g., hemangiomas, acoustic neuromas, neurofibromas, trachomas, and pyogenic granulomas; rheumatoid arthritis); psoriasis; ocular angiogenic diseases, for example, diabetic retinopathy, retinopathy of prematurity, macular degeneration, corneal graft rejection, neovascular glaucoma, retrolental fibroplasia, rubeosis; Osler-Webber Syndrome; myocardial angiogenesis; plaque neovascularization; telangiectasia; hemophiliac joints; angiofibroma; and wound granulation.

“Angiogenesis-dependent cancers and tumors” are cancers and tumors that require, for their growth (expansion in volume and/or mass), an increase in the number and density of the blood vessels supplying then with blood. The plasmin inhibitor can be used in treatment to cause regression of such cancers and tumors. “Regression” refers to the reduction of tumor mass and size, e.g., a reduction of at least 2, 5, 10, or 25%.

A plasmin inhibitor, such as a protein comprising a Kunitz domain, e.g., DX-1000 or other protein described herein, can be administered as an adjuvant therapy, e.g., to a subject. The adjuvant therapy can be a post-operative therapy that is administered to the subject after the subject has undergone surgery to remove all or part of a tumor (e.g., after surgery to treat angiogenesis-dependent cancer). In one embodiment, the plasmin inhibitor is administered within 6, 12, 24, 48, or 100 hours of surgery. The plasmin inhibitor can be administered before, during, as well as after surgery.

### Treating Lymphangiogenesis-Related Disorders

VEGF-C and VEGF-D stimulate lymphangiogenesis and angiogenesis in tissues and tumors by activating the VEGF receptor VEGFR-2 and VEGFR-3. These growth factors are secreted as full-length inactive forms consisting of NH2- and COOH-terminal propeptides and a central VEGF homology domain containing receptor binding sites. Proteolytic cleavage removes the propeptides to generate mature forms, consisting of dimers of the VEGF homology domain that bind receptors with much greater affinity than the full-length forms. Plasmin cleaves both propeptides from the VEGF homology domain of human VEGF-D and thereby generates a mature form exhibiting greatly enhanced binding and cross-linking of VEGFR-2 and VEGFR-3 in comparison to full-length material. Plasmin also activates VEGF-C. As lymphangiogenic growth factors promote the metastatic spread of cancer via the lymphatics, the proteolytic activation of these molecules represents a potential target for antimetastatic agents. The plasmin inhibitor can be used in treatment as an anti-metastatic agent especially in breast, ovarian, and colorectal cancers where both VEGF-C and VEGF-D are highly expressed and associated with lymph node metastasis (Nakamura et al., 2003, Mod. Pathol. 16:309-314; Yokoyama et al. 2003. Br. J. Cancer. 88:237-244; White et al., 2002, Cancer Res. 62:1669-1675; Nakamura et al., 2003. Clin. Cancer Res. 9:716-721).

A plasmin inhibitor, e.g., a protein that includes a Kunitz domain, e.g., DX-1000 or other protein described herein, can be used to lymphangiogenesis-related disorders, e.g., cancer, e.g., metastatic breast, ovarian, or colorectal cancer. In one embodiment, treatment for lymphangiogenesis-related disorder may include a combination of at least two therapies, for example, administering a plasmin inhibitor, e.g., one described herein, in combination with a second therapy. Examples of a second therapy include surgery, radiation therapy, and hormonal therapy.

In another aspect, the disclosure features a method of reducing VEGF-C and/or VEGF-D activity in a subject. In one embodiment, the method includes administering, to a human or animal subject, a plasmin-inhibitory amount of a protein including a Kunitz domain that inhibits plasmin, e.g., DX-1000.

In another aspect, the disclosure features administering a plasmin inhibitor such as a protein comprising a Kunitz domain, e.g., DX-1000 or other protein described herein, as an adjuvant therapy, e.g., to a subject. The adjuvant therapy can be a post-operative therapy that is administered to the subject after the subject has undergone surgery to remove all or part of a lymphangiogenesis-related tumor (e.g., after surgery to treat breast, ovarian, or colorectal cancer). In one embodiment, a plasmin inhibitor, e.g., a protein comprising a Kunitz domain that inhibits plasmin is administered within 6, 12, 24, 48, or 100 hours of surgery. The plasmin inhibitor can be administered before, during, as well as after surgery.

### Example 1

We observed that DX-1000 (i) inhibited plasmin-mediated MMP activation, (ii) decreased in vitro invasiveness of tumor cells, (iii) decreased in vitro angiogenesis, (iv) did not inhibit migration, and (v) did not significantly influence haemostasis in vitro. These properties indicate that DX-1000 and similar plasmin inhibitors can be used to treat and prevent cancers, particularly metastatic cancers.

### Methods

Cell culture of tumor cell lines: HT-1080 (fibrosarcoma), and LNCaP (androgen-dependent prostate cancer) cell lines were grown to 80% confluence in Dulbecco's modified Eagle medium (DMEM) supplemented with 10% FCS, glutamine (292 mg/ml), sodium bicarbonate (2.1 g/l), ascorbic acid (50 g/ml) and penicillin-streptomycin (P/S) (100 U/ml). LNCaP cells were stimulated with di-hydro-testosterone (DHT, 10 nM). Non-adherent HL-60 (acute myeloid leukemia) cell line was grown in RPMI medium supplemented with 20% FCS, glutamine 4 mM, sodium bicarbonate (1.5 g/l) and P/S (100 U/ml). Cultures were maintained at a cell concentration between 1e5 and 1e6/ml.

Gelatin zymography: HL-60 cells were cultured in serum-free medium (Ultraculture medium). Different conditions were considered: with and w/o DX-1000 at 1 μM; with or without pro-MMP-9 activators (plasmin+pro-MMP-3). Cells were cultured at 37° C./5% CO₂ for 48 hours. Aliquots of conditioned media (CM) were then collected, clarified by centrifugation, mixed with electrophoresis sample buffer without reducing agent, and applied to 10% acrylamide gels containing gelatin (1 mg/ml). Active recombinant gelatinases served as standard. Samples were resolved at 20 mA, washed in 2% Triton X-100 for 1 hour and incubated at 37° C. for 16 hours in activation buffer containing 50 mM Tris-HCl, pH 7.4, 0.2 M NaCl, 5 mM CaCl₂. After staining with Coomassie brilliant blue R-250, the gelatinolytic activities were detected as clear bands against the blue background.

Chemo-invasion: HT-1080 and LNCaP cells were seeded at 10⁶/well in 24-well cluster plates and incubated overnight. They were then incubated in the presence of a dose range of DX-1000 for 24 hours. The well-known plasmin inhibitor, aprotinin, (TRASYLOL®) and TIMP-4 were used as controls. Chemo-invasion was assessed using Transwell cell culture chamber inserts (Becton Dickinson) with Growth Factor Reduced-MATRIGEL® (GFR-MATRIGEL®) coated filters. After trypsinization, cells were seeded in the upper part of the invasion chamber (1e4/insert). RPMI medium supplemented with 5% FCS and 1% BSA and NIH3T3 CM used as chemoattractants for HT-1080 and LNCaP cells, respectively. After 24 hours of incubation at 37° C., filters were removed from the chambers, stained, and the % of invading cells was evaluated. Each condition was performed in duplicate.

Tube formation assay: HUVECs were cultured on gelatin-coated culture dishes in RPMI medium. The cells (passage 2) were seeded at 8e4/well of a 96-well plate on collagen type I (1.5 mg/ml, SERVA) in their culture medium (EGM-2 complete medium supplemented with 10% FCS) and allowed to spread for 1 hour. The culture medium was then discarded and the cells were covered with a new layer of collagen type I (1.5 mg/ml, new preparation). After polymerization of the gel, culture medium was added to each well in the presence or absence of DX-1000 (1 nM to 10 μM) or aprotinin and incubated at 37° C./5% CO₂ for 16-18 hours. Mouse endothelial cells (EC) (LEII cell line) were seeded at 2e4/well of a 96-well plate on GFR-Matrigel (BD) in their culture medium (IMDM supplemented with 10% FCS) in presence of a dose range of DX-1000 and incubated at 37° C./5% CO₂ for 5 hours. Endothelial tube formation was assessed with an inverted photomicroscope (Analis). Microphotographs of the center of each well at low power (40×) were taken with a NIKON camera with the aid of imaging-capture software (NIKON). Tube formation in the microphotographs was quantitatively analyzed (total tube length) with METAVUE™ software (Universal Imaging Corporation). Tube formation by untreated HUVECs in full endothelial cell growth medium was used as a control. IC50 values were determined with SIGMAPLOT™.

We observed that the IC₅₀ value of inhibiting tube formation by HUVECs was about 1.4 (+/−) 0.3 nM.

Scraping assay: HT-1080 cells were seeded at 2e6/well in 6-well plates in complete medium. Confluent cells were incubated in the presence of DX-1000 or Aprotinin (1-10 μM) for 5 hours. Cell layers were then scraped with a plastic tip. Pictures were taken every hour for 6 hours, corresponding to the time necessary for the non-treated cells to fully recover the scraping area.

### Results

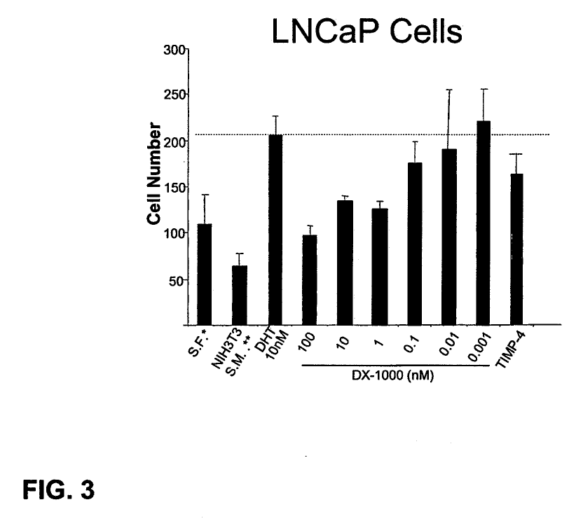

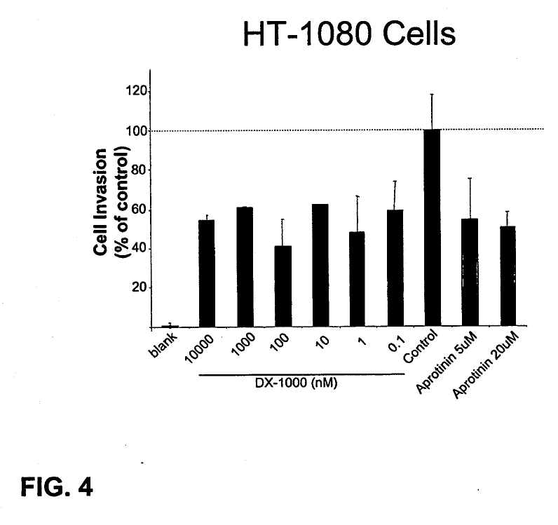

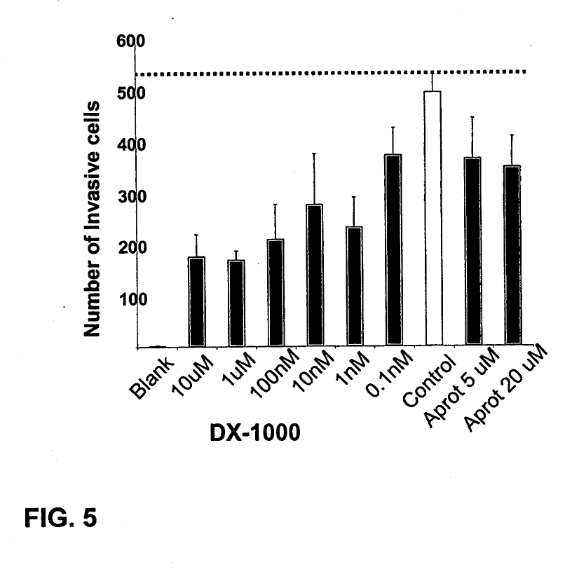

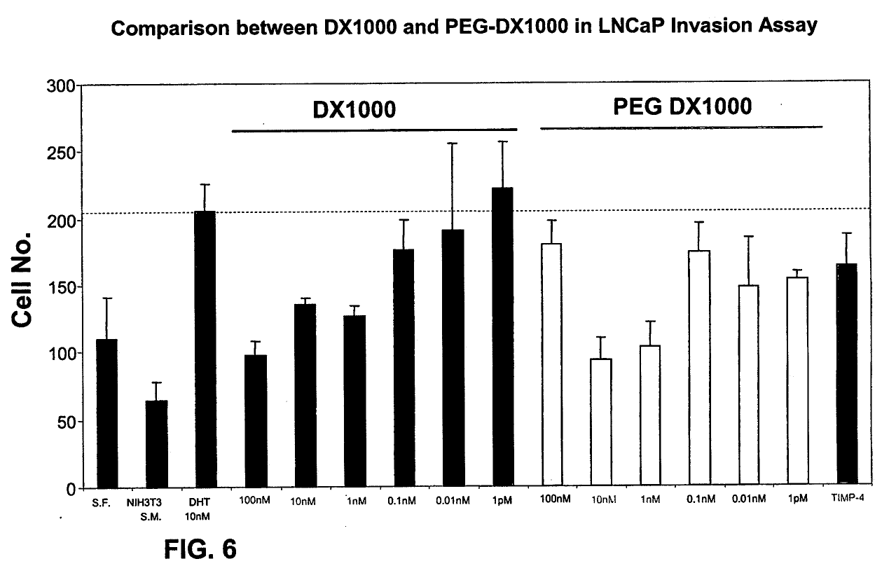

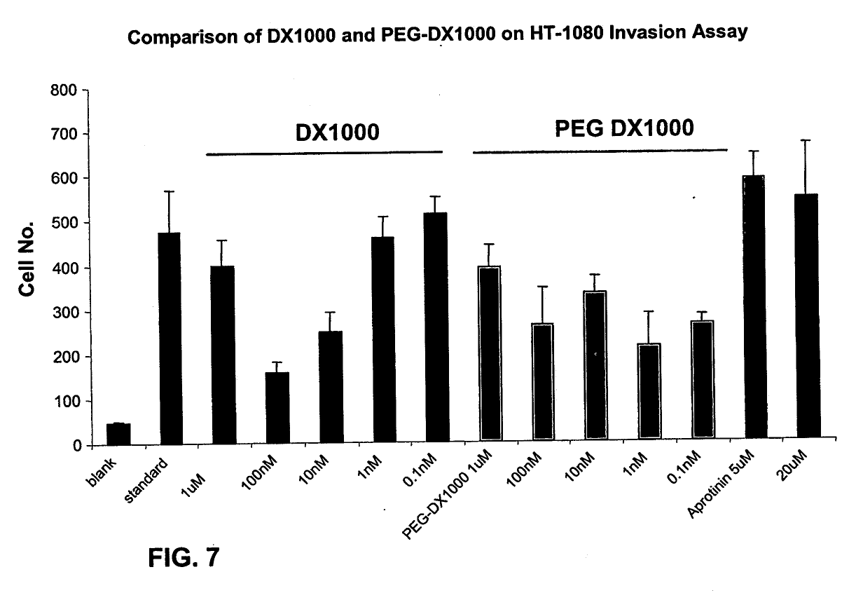

We observed that DX-1000 decreases the in vitro invasiveness of tumor cells. See, e.g., FIGS. 3-7. Invasion was evaluated using HT-1080 cells (a fibrosarcoma) and LNCaP (androgen-dependent prostate cancer). DX-1000 was effective at nanomolar and sub-nanomolar concentrations at inhibiting invasion in vitro. DX-1000 was also a more potent inhibitor than aprotinin in these assays.

We also observed that DX-1000 down-regulates MMP expression and activation. Using gelatin zymography, we observed that DX-1000 inhibits plasmin-mediated pro-MMP-9 activation in HL-60 cells (64% inhibition). Similar results were observed for 4-PEG DX-1000 (92% inhibition).

In addition to gelatin zymography, we evaluated the plasmin inhibitors using the BIOTRAK® assay kit. HL60 were seeded at 4×10⁵ cells/well (24 wells plate) in a serum free medium (UltraCulture medium). The day after, different conditions were evaluated: samples with and without activators of pro-MMP9 (Plasmin and pro-MMP-3) and samples with and without DX-1000 and 4-PEG-DX-1000 (1 μM). After two days of culture, conditioned media were collected an activity of MMP-9 was assessed using the BIOTRAK® assay kit. Assay time was approximately one hour under standard conditions. At a 1 μM concentration, DX-1000 and 4-PEG DX-1000 effected an approximately 68-70% reduction in plasmin-activated MMP-9 activity.

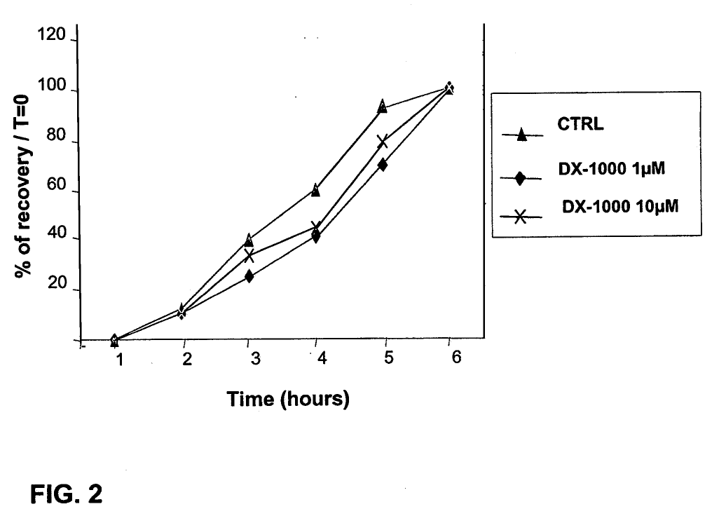

The effect of DX-1000 on cell migration was evaluated using two different assays, the scraping assay and a Boyden chamber assay without Matrigel. DX-1000 does not inhibit the two-dimensional migration of HT-1080 cells in vitro (FIG. 2). Similar results were observed for aprotinin.

DX-1000 was also tested for its activity in an in vitro endothelial tubulogenesis assay (“tube formation assay” described in Methods.). We observed that it is a potent inhibitor of tubulogenesis in vitro and is at least as efficient as aprotinin at inhibiting tube formation. 4-PEG DX-1000 also inhibited tube formation. The IC₅₀ values observed were as follows:

| Protein | IC50 (nM) |
|---|---|
| DX-1000 (unmodified) | HUVEC: 1.39 ± 0.28 |
| | Mouse EC: 16.6 ± 0.1 |
| 4-PEG DX-1000 (batch #1, small scale) | HUVEC: 8.3 ± 1.6 |
| | Mouse EC: 15.8 ± 0.6 |
| 4-PEG DX-1000 (batch #2, large scale) | 0.98 ± 1.25 |

We also evaluated DX-1000 in an assay to measure the sensitivity of human tumors to drugs before progressing to in vivo studies. SW-480 cells are grown in vitro in soft agar, reducing cell movement and allowing individual cells to develop into cell clones that are identified as single colonies. 3,000 SW480 cells were seeded into each well of six well plates. Each treatment was run in triplicate. Five 4× images were taken from each well for quantification using the METAVIEW® software. Both DX-1000 and 4-PEG DX-1000, at concentrations ranging from 0.1 to 50 μM, did not inhibit colony formation by SW480 cells (whereas cisplatin at 33 μM did).

DX-1000 and 4-PEG DX-1000 do not induce apoptosis in HL-60 and SW-480 cells. Apoptosis was evaluated using an assay for detecting caspase 3/7 activity using caspase 3/7 substrate. Apoptosis was also not detected using a FACS assay.

We also did not detect any clinically significant effects on DX-1000 in global coagulation screening tests. Low concentrations of DX-1000 can be used without inhibiting clot lysis. Inhibition of clot lysis was observed with concentrations of DX-1000>700 nM.

Thrombelastography (TEG), a global method for evaluation of haemostasis, provided evidence of inhibition of fibrinolysis by 280-560 nM DX-1000 in two subjects with upper normal values. tPA: reduction in fibrinolysis with 280-560 nM DX-1000.

DX-1000 showed a weak dose-dependent effect on activated partial thromboplastin time (APTT) at higher doses (1.4-5.6 μM). Clotting times remained within the normal range. We did not observe an effect of DX-1000 on prothrombin time (PT), Clauss fibrinogen assays at concentrations>5.6 μM.

### Example 2

The following pharmacokinetic studies show plasma clearance, stability and the biodistribution of DX-1000 and 4PEG-DX-1000 in normal mice and rabbits.

### Methods:

Labeling: we used 500-700 mg protein and the Iodogen (Pierce) method, ˜1.7 mCi.
Purification: we used D-salt polyacrylamide column and collected fractions, with total recovery ˜90%. We pooled high yield fractions for injections.
Mice: we injected 5 mg/animal via tail vein. One time point/animal; four animals/time point. We used the following time points: no PEG at 0, 7, 15, 30, and 90 min. PEG at 0, 7, 15, 30, and 90 min, 4, 8, 16, and 24 hrs. We analyzed total plasma counts, used HPLC on Superose 12 (stability), and analyzed biodistribution.
Rabbits: we injected 80 mg/animal via left ear vein. Blood was drawn from the right ear at following time points: no PEG at 0, 7, 15, 30, 90 min, 4, 8, 16, 24, 48, 72, and 96 hrs. PEG at 0, 7, 15, 30, 90 min, 4, 8, 16, 24, 48, 72, 96, 120, 144, 168, and 192 hrs. We preformed the following analyses: total plasma counts and HPLC on Superose 12 (stability) at 48, 72, 96, 120, 144, 168, and 192 hrs.

### Results

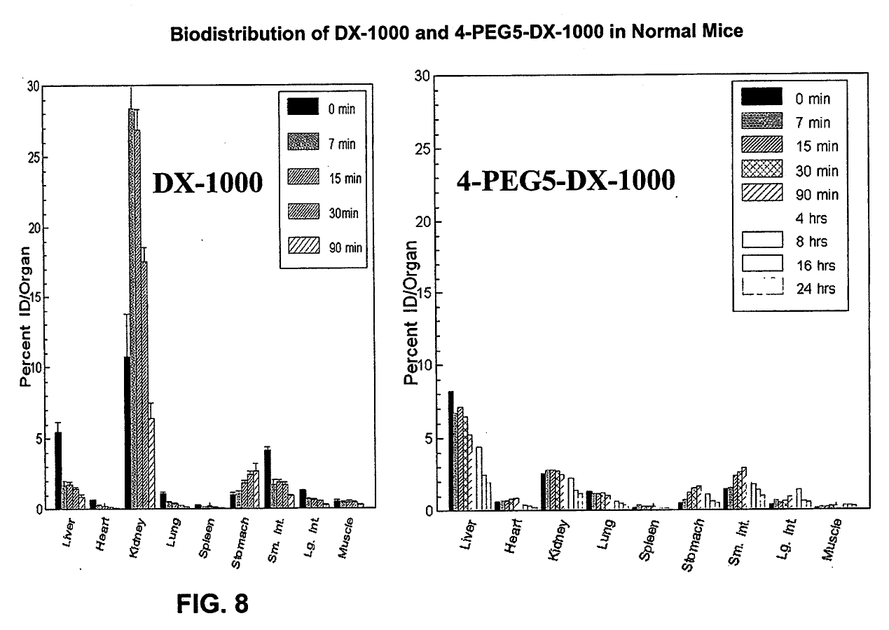

In vivo pharmacokinetic studies in mice with ionidated DX-1000 and 4PEG-DX1000 show a significant increase of half-life between DX-1000 and its PEGylated derivative (DX-1000: 0.45 h; 4PEG-DX1000: 13 h). FIG. 8 shows the results of biodistribution studies in normal mice.

In vivo pharmacokinetic studies in rabbit with ionidated DX-1000 and 4PEG-DX1000 show a significant increase of half-life between DX-1000 and its PEGylated derivative (DX-1000: about 1 h; 4PEG-DX1000: 59 h). By allometric extrapolations, we should expect a stability of 9 days in humans.

### Example 3

The following in vitro experiments can be used to evaluate proteins for their ability to modulate tumor invasion. Examples of proteins that can be evaluated include plasmin inhibitors such as DX-1000, pegylated DX-1000, and DX-1000 fused to albumin, or fragment thereof.

DX-1000 can be tested in MDA-MB-231 (human breast cancer cells) and PC-3 (human prostate cancer cells) tumor cell invasion assay. These assays can be carried out according to established protocols of Matrigel invasion assay. Vehicle control, aprotinin, and three dilutions of DX-1000 can be tested in triplicate for evaluating the ability of DX-1000 to alter tumor cell invasive capacity. These studies can be repeated three times.

Also, three concentrations of DX-1000 can be tested in MDA-MB-231 and PC-3 tumor cell invasion and migration assay. Vehicle control and DX-1000 can be tested in triplicate. These experiments can be repeated three times.

### Example 4

The following in vivo experiments can be used to evaluate proteins for their ability to modulate tumor growth and invasion. Examples of proteins that can be evaluated include plasmin inhibitors such as DX-1000, pegylated DX-1000, and DX-1000 fused to albumin, or fragment thereof.

Human breast cancer cells MDA-MB-231 transfected with green fluorescent protein (MDA-MB-231-GFP) can be inoculated into the mammary fat pad of female BALB.c nu/nu mice. Animals can be monitored for tumor growth. At week 4-5 post tumor cell inoculation animals with tumors of 3-50 mm³ can be selected, randomized and divided into four groups. Animals can be treated with vehicle alone or two different doses of DX-1000. An appropriate positive control (DOX, Taxotere) can be included in one arm of the study. Dosages, route of administration and frequency can be determined, e.g., following guidance from animal models and in vitro studies.

Female BALB.c nu/nu mice can be inoculated with human prostate cancer PC-3-GFP cells into their tibia or left ventricle. Animals can be monitored for tumor growth by weekly radiological (Faxitron) analysis. Animals can be treated with vehicle alone or two different doses of DX-1000. An appropriate positive control (DOX, Taxotere) can be included in one arm of the study.

Tissue analysis:

1: At the end of all above described studies primary tumors and different organs can be removed from at least 6 animals/group for the detection and quantification of microscopic tumor metastasis.

2: All animals in the PC-3-GFP study can be subjected to radiological analysis at regular intervals.

3: At the end of these studies, animals can be sacrificed and representative long bones will be analyzed by micro CT analysis.

4: Representative (6 per group) primary tumors and long bones can be removed and subjected to histological and bone histomorphometric analysis to determine tumor volume to tissue volume ratios.

5: Immunohistochemical analysis of primary tumors and long bones can be carried out to evaluate change in the expression of various genes (uPA, plasminogen, CD31, Ki67) involved in tumor progression following treatment with DX-1000.

Other embodiments are within the following claims.

1. (canceled)

2. A method of treating a breast cancer or a breast cancer-derived metastasis, the method comprising:
administering, to a subject who has or is suspected of having breast cancer, an effective amount of a protein comprising a Kunitz domain that comprises the binding loops of DX-1000 or loops that differ by two or fewer amino acids from the binding loops of DX-1000.

3.-5. (canceled)

6. The method of claim 2, wherein the breast cancer or the breast cancer-derived metastasis is an angiogenesis-dependent cancer or a tumor.

7. The method of claim 2, wherein the breast cancer highly expresses VEGF-C and VEGF-D.

8. The method of claim 2, wherein the protein is administered intravenously.

9. The method of claim 2, wherein the Kunitz domain is mono-PEGylated.

10. The method of claim 2, wherein the Kunitz domain is poly-PEGylated.

11. The method of claim 2, wherein the Kunitz domain is fused to an albumin, or a fragment thereof.

12. The method of claim 11, wherein the protein is administered in combination with a second therapy.

13. The method of claim 2, wherein the effective amount of the protein does not impair coagulation or platelet function.

14. The method of claim 2, wherein the protein is administered as part of a post-operative adjuvant therapy, to a subject who has had surgery to remove a tumor.

15. The method of claim 2, wherein the Kunitz domain differs from DX-1000 by fewer than 3 amino acid differences.

16. The method of claim 2, wherein the Kunitz domain comprises the sequence of SEQ ID NO:22.

17. The use according to claim 16, wherein the Kunitz domain comprises the sequence of SEQ ID NO:23.

18. The use according to claim 12, wherein the protein is administered in combination with a plasma kallikrein inhibitor.


SEQUENCE LISTING (24 sequences)

SEQ ID NO: 1 - protein, 304 aa, Homo sapiens
MIYTMKKVHALWASVCLLLNLAPAPLNADSEEDEEHTIITDTELPPLKLMHSFCAFKADDGPCKAIMKRFFFNIFTRQCEEFIYGGCEGNQNRFESLEECKKMCTRDNANRIIKTTLQQEKPDFCFLEEDPGICRGYITRYFYNNQTKQCERFKYGGCLGNMNNFETLEECKNICEDGPNGFQVDNYGTQLNAVNNSLTPQSTKVPSLFEFHGPSWCLTPADRGLCRANENRFYYNSVIGKCRPFKYSGCGGNENNFTSKQECLRACKKGFIQRISKGGLIKTKRKRKKQRVKIAYEEIFVKNM

SEQ ID NO: 2 - protein, 58 aa, Bos taurus [sequence omitted]

SEQ ID NO: 3 - protein, 58 aa, Artificial Sequence
  Synthetically generated peptide
SDDPCSLPLDEGSCTAYTLRWYHRAVTEACHPFVYGGCGGNANRFGTREACERRCPPR

SEQ ID NO: 4 - protein, 58 aa, Artificial Sequence
  Synthetically generated peptide
NAEICLLPLDYGPCRALLLRYYYDRYTQSCRQFLYGGCEGNANNFYTWEACDDACWRI

SEQ ID NO: 5 - protein, 58 aa, Artificial Sequence
  Synthetically generated peptide
VREVCSEQAETGPCRAMISRWYFDVTEGKCAPFFYGGCGGNRNNFDTEEYCMAVCGSA

SEQ ID NO: 6 - protein, 58 aa, Artificial Sequence
  Synthetically generated peptide
YEEYCTANAVTGPCRASFPRWYFDVERNSCNNFIYGGCRGNKNSYRSEEACMLRCFRQ

SEQ ID NO: 7 - protein, 58 aa, Artificial Sequence
  Synthetically generated peptide
KEDSCQLGYSAGPCMGMTSRYFYNGTSMACETFQYGGCMGNGNNFVTEKECLQTCRTV

SEQ ID NO: 8 - protein, 59 aa, Artificial Sequence
  Synthetically generated peptide
FQEPCMLPVRHGNCNHEAQRWHFDFKNYRCTPFKYRGCEGNANNFLNEDACRTACMLIR

SEQ ID NO: 9 - protein, 56 aa, Artificial Sequence
  Synthetically generated peptide
TEDYCLNKVGRCRGSFPRWYYDPTEQICKSFVYGGCLGNKNNYLREEECILACRGV

SEQ ID NO: 10 - protein, 58 aa, Artificial Sequence
  Synthetically generated peptide
DKGHCVDLPDTGLCKESIPRWYYNPFSEHCARFTYGGCYGNKNNFEEEQQCLESCRGI

SEQ ID NO: 11 - protein, 56 aa, Artificial Sequence
  Synthetically generated peptide
IPSFCPKDEGLCSANVTRYYFNPRYRTCDAFTYTGCGGNDNNFVSREDCKRACAKA

SEQ ID NO: 12 - protein, 56 aa, Artificial Sequence
  Synthetically generated peptide
AACNLPIVRGPCRAFIQLWAFDAVKGKCVLFPYGGCQGNGNKFYSEKECREYCGVP

SEQ ID NO: 13 - protein, 58 aa, Artificial Sequence
  Synthetically generated peptide
IHDFCLVSKVVGRCRASMPRWWYNVTDGSCQLFVYGGCDGNSNNYLTKEECLKKCATV

SEQ ID NO: 14 - protein, 58 aa, Artificial Sequence
  Synthetically generated peptide
VKAVCSQEAMTGPCRAVMPRWYFDLSKGKCVRFIYGGCGGNRNNFESEDYCMAVCKAM

SEQ ID NO: 15 - protein, 58 aa, Artificial Sequence
  Synthetically generated peptide
KPDFCFLEEDPGICRGYITRYFYNNQTKQCERFKYGGCLGNMNNFETLEECKNICEDG

SEQ ID NO: 16 - protein, 61 aa, Artificial Sequence
  Synthetically generated peptide
VPKVCRLQVSVDDQCEGSTEKYFFNLSSMTCEKFFSGGCHRNRIENRFPDEATCMGFCAPK

SEQ ID NO: 17 - protein, 58 aa, Artificial Sequence
  Synthetically generated peptide
LPNVCAFPMEKGPCQTYMTRWFFNFETGECELFAYGGCGGNSNNFLRKEKCEKFCKFT

SEQ ID NO: 18 - protein, 58 aa, Artificial Sequence
  Synthetically generated peptide
MHSFCAFKADDGPCKAIMKRFFFNIFTRQCEEFIYGGCEGNQNRFESLEECKKMCTRD

SEQ ID NO: 19 - protein, 58 aa, Artificial Sequence
  Synthetically generated peptide
GPSWCLTPADRGLCRANENRFYYNSVIGKCRPFKYSGCGGNENNFTSKQECLRACKKG

SEQ ID NO: 20 - protein, 58 aa, Artificial Sequence
  Synthetically generated peptide
ETDICKLPKDEGTCRDFILKWYYDPNTKSCARFWYGGCGGNENKFGSQKECEKVCAPV

SEQ ID NO: 21 - protein, 58 aa, Artificial Sequence
  Synthetically generated peptide
KQDVCEMPKETGPCLAYFLHWWYDKKDNTCSMFVYGGCQGNNNNFQSKANCLNTCKNK

SEQ ID NO: 22 - protein, 58 aa, Artificial Sequence
  Synthetically generated peptide
MHSFCAFKAETGPCRARFDRWFFNIFTRQCEEFIYGGCEGNQNRFESLEECKKMCTRD

SEQ ID NO: 23 - protein, 60 aa, Artificial Sequence
  Artificial Kunitz Domain
EAMHSFCAFKAETGPCRARFDRWFFNIFTRQCEEFIYGGCEGNQNRFESLEECKKMCTRD

SEQ ID NO: 24 - protein, 58 aa, Artificial Sequence
  Synthetically generated peptide
XXXXCXXXXXXGXCXXXXXRXXXXXXXXXCXXFXXXGCXXXXXXXXXXXXCXXXCXXX